(19) United States
(12) Patent Application Publication
(10) Pub. No.: US 2004/0010005 A1
(43) Pub. Date: Jan. 15, 2004

(54) Stereoselective process for preparing cylcohexyl amine derivatives

(76) Inventors: Charles Jackson Barnett, Indianapolis, IN (US); Rui Lin Gu, Indianapolis, IN (US); Michael Edward Kobierski, Greenwood, IN (US)

Correspondence Address: ELI LILLY AND COMPANY, PATENT DIVISION, P.O. BOX 6288, INDIANAPOLIS, IN, 46206-6288, US

(21) Appl. No.: 10/362,496

(22) PCT Filed: Sep. 13, 2001
(86) PCT No.: PCT/US01/26023

(51) Int. Cl. C07D 491/02; A61K 31/4745

(52) U.S. Cl. 514/292; 546/83

(57) ABSTRACT

A process for preparing (1R,3S)-3-(9-chloro-3-methyl-4-oxo-5H-(isoxazoloquinolin-5-yl))cyclohexanecarboxylic acid and esters thereof, as represented by formulas II and III, wherein R is a lower alkyl groups, and A and B are N or O, provided that when A is N, B is O, or when A is O, B is N: Formula (III) and Formula (II).

Along with surgery and radiotherapy, chemotherapy continues to be an effective therapy for many cancers. In fact, several types of cancer, such as Hodgkin's disease, large cell lymphoma, acute lymphocytic leukemia, testicular cancer and early stage breast cancer, are now considered to be curable by chemotherapy. Other cancers such as ovarian cancer, small cell lung and advanced breast cancer, while not yet curable, are exhibiting positive response to combination chemotherapy.

One of the most important unsolved problems in cancer treatment is drug resistance. After selection for resistance to a single cytotoxic drug, cells may become cross resistant to a whole range of drugs with different structures and cellular targets, e.g., alkylating agents, antimetabolites, hormones, platinum-containing drugs, and natural products. This phenomenon is known as multidrug resistance (MDR). In some types of cells, this resistance is inherent, while in others, such as small cell lung cancer, it is usually acquired.

Such resistance is known to be multifactorial and is conferred by at least two proteins: the 170 kDa P-glycoprotein (MDR1) and the more recently identified 190 kDa multidrug resistance protein (MRP1). Although both MDR1 and MRP1 belong to the ATP-binding cassette superfamily of transport proteins, they are structurally very different molecules and share less than 15% amino acid homology. Despite the structural divergence between the two proteins, by 1994 there were no known consistent differences in the resistance patterns of MDR1 and MRP1 cell lines. However, the association, or lack thereof, of MRP1 and resistance to particular oncolytics is known. See Cole, et. al., “Pharmacological Characterization of Multidrug Resistant MRP-transfected Human Tumor Cells”, Cancer Research, 54:5902-5910, 1994.

Doxorubicin, daunorubicin, epirubicin, vincristine, and etoposide are substrates of MRP1, i.e., MRP1 can bind to these oncolytics and redistribute them away from their site of action, the nucleus, and out of the cell. Id. and Marquardt, D., and Center, M. S., Cancer Research, 52:3157, 1992.

Doxorubicin, daunorubicin, and epinubicin are members of the anthracycline class of oncolytics. They are isolates of various strains of Streptomyces and act by inhibiting nucleic acid synthesis. These agents are useful in treating neoplasms of the bone, ovaries, bladder, thyroid, and especially the breast. They are also useful in the treatment of acute lymphoblastic and myeloblastic leukemia, Wilm's tumor, neuroblastoma, soft tissue sarcoma, Hodgkin's and non-Hodgkin's lymphomas, and bronchogenic carcinoma.

Vincristine, a member of the vinca alkaloid class of oncolytics, is an isolate of a common flowering herb, the periwinkle plant (Vinca rosea Linn). The mechanism of action of vincristine is still under investigation but has been related to the inhibition of microtubule formation in the mitotic spindle. Vincristine is useful in the treatment of acute leukemia, Hodgkin's disease, non-Hodgkin's malignant lymphomas, rhabdomyosarcoma, neuroblastoma, and Wilm's tumor.

Etoposide, a member of the epipodophyllotoxin class of oncolytics, is a semisynthetic derivative of podophyllotoxin. Etoposide acts as a topoisomerase inhibitor and is useful in the therapy of neoplasms of the testis, and lung.

It is presently unknown what determines whether a cell line will acquire resistance via a MDR1 or MRP1 mechanism. Due to the tissue specificity of these transporters and/or in the case where one mechanism predominates or is exclusive, it would be useful to have a selective inhibitor of that one over the other. Furthermore, when administering a drug or drugs that are substrates of either protein, it would be particularly advantageous to coadminister an agent that is a selective inhibitor of that protein. It is, therefore, desirable to provide compounds that are selective inhibitors of MDR1 or MRP1, which can be manufactured by large scale processing without compromising stereoselectivity, purity, and yield.

The present invention provides a process for preparing (1R,3S)-3-(9-chloro-3-methyl-4-oxo-5H-(isoxazoloquinolin-5-yl))cyclohexanecarboxylic acid and esters thereof, as represented by formulas II and m, wherein R is a lower alkyl group, benzyl, aryl, or heterocycle, and A and B are N or O, provided that when A is N, B is O, or when A is O, B is N:

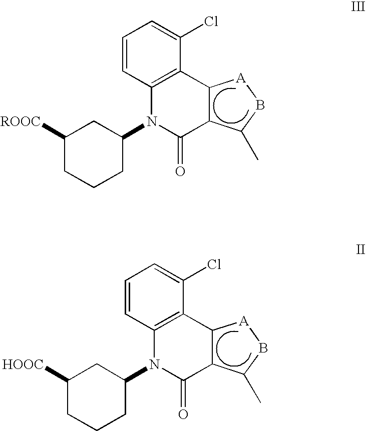

comprising the steps of:

(a) treating, in an appropriate solvent, a compound of formula (i), wherein R is as defined above:

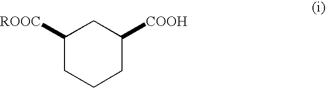

with an appropriate azide and subsequently an appropriate alcohol to form a compound of formula (ii), wherein R is defined above and R′ is a lower alkyl group, benzyl, aryl, or heterocycle:

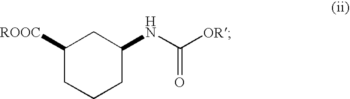

(b) deprotecting the compound of formula (ii), in an appropriate reaction medium, with an appropriate deprotecting agent to provide a compound of formula (iii), wherein R is as defined above:

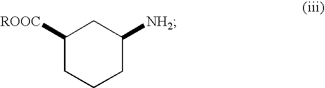

(c) reacting the compound of formula (iii), in an appropriate solvent, with (6-chloro-2-fluorophenyl)-methylisoxazole-4-carbonyl chloride to provide the compound of formula (iv), wherein R, A, and B are as defined above:

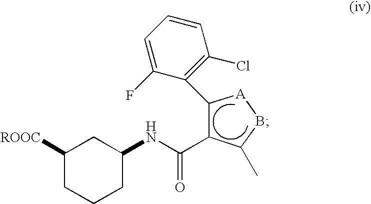

(d) cyclizing the compound of formula (iv), in an appropriate solvent, in the presence of an appropriate catalyst, to form a compound of formula III, wherein R, A, and B are as defined above:

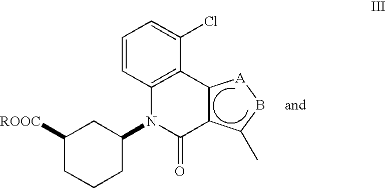

(e) optionally, hydrolyzing the compound of formula III to form a compound of formula II, wherein R, A, and B are as defined above:

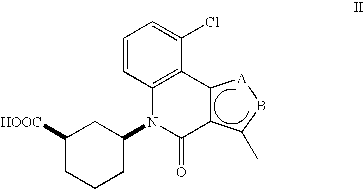

Furthermore, the invention relates to a process for converting the compounds of formulas II or III to form a compound of formula I(a):

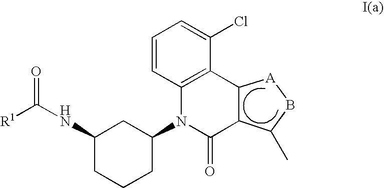

wherein:

the stoichiometry is substituted (1R,3S)-3-(9-chloro-3-methyl-4-oxo-5H-(isoxazoloquinolin-5-yl) cyclohexane;

a is 0, 1, 2, 3, or 4;

b is 0, 1, or 2;

u is 0, 1, 2, 3, or 4;

A is N or O; when A is N, B is O, or when A is O, B is N;

R¹ is independently at each occurrence C₁-C₆ alkyl, C₁-C₆ alkoxy, (C₁-C₄ alkoxy)-aryl, (C₁-C₄ alkoxy)-heterocycle,(C₁-C₄ alkoxy)-SiCH₃, optionally substituted (C₁-C₄ alkyl)-(C₃-C₈ cycloalkyl),optionally substituted (C₁-C₄ alkyl)-aryl, optionally substituted aryl, diphenylmethyl, optionally substituted (C₁-C₄ alkyl)-CO-aryl, optionally substituted (C₁-C₄ alkyl)-heterocycle, optionally substituted heterocycle, optionally substituted (C₁-C₄ alkyl)-phenoxy, (CH₂)ₐS(O)_{b}R², (CH₂)ₐC(R³)(R⁴)N(R⁵)(R⁶), (CH₂)ₐC(R³)(R⁴)O(R⁷), (CH₂)ₐC(R³)(R⁴)S(R⁷), or NR⁸R⁹;

R² is independently at each occurrence hydrogen or C₁-C₆ alkyl;

R³ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, or optionally substituted heterocycle;

R⁴ is independently at each occurrence hydrogen, C₁-C₆ alkyl, optionally substituted C₃-C₈ cycloalkyl, optionally substituted (C₁-C₄ alkyl)-aryl, optionally substituted aryl, optionally substituted heterocycle, (CH₂)ᵤ-(C₁-C₆ alkoxy), optionally substituted (CH₂)ᵤ-O-(C₃-C₈ cycloalkyl), optionally substituted (CH₂)ᵤ-(C₁-C₄ alkoxy)-aryl, optionally substituted (CH₂)ᵤ-O-aryl, optionally substituted (CH₂)ᵤ-O-heterocycle, (C₁-C₄ alkyl)-CO₂-(C₁-C₆ alkyl), optionally substituted (C₁-C₄ alkyl)-CO₂-(C₃-C₈ cycloalkyl), optionally substituted (C₁-C₄ alkyl)-CO₂-(C₁-C₄ alkyl)-aryl, optionally substituted (C₁-C₄ alkyl)-CO₂-aryl, optionally substituted (C₁-C₄ alkyl)-CO₂-heterocycle, or R9 and R12 can combine to form a C₃-C₈ cycloalkyl;

R⁵ is independently at each occurrence hydrogen, optionally substituted C₁-C₆ alkyl, optionally substituted aryl, optionally substituted heterocycle, or -COR¹¹; or R⁵ and R⁶, together with the nitrogen to which they are attached, combine to form an optionally substituted N-heterocycle;

R⁶ is independently at each occurrence hydrogen, C₁-C₆ alkyl, optionally substituted C₃-C₈ cycloalkyl, optionally substituted C₆-C₁₀ bicycloalkyl, optionally substituted (C₁-C₄ alkyl)-aryl, optionally substituted aryl, optionally substituted (C₁-C₄ alkyl)-heterocycle, optionally substituted heterocycle, C(O)OR¹⁰, SO₂R¹¹, C(O)R¹², or a moiety of the formula

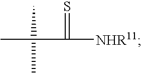

R⁷ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, optionally substituted heterocycle, optionally substituted (C₁-C₄ alkyl)-heterocycle, optionally substituted C₁-C₆ alkoxy, optionally substituted (C₁-C₄ alkoxy)-aryl, optionally substituted (C₁-C₄ alkoxy)-heterocycle, (C₁-C₄ alkyl)-N(R²)(R²),or an amino acid ester;

R⁸ is independently at each occurrence hydrogen, C₁-C₆ alkyl, optionally substituted (C₁-C₆ alkyl)-aryl, optionally substituted aryl, or R⁸ and R⁹ combine to form ═CR²R¹³;

R⁹ is independently at each occurrence hydrogen, C₁-C₆ alkyl, C₁-C₄ alkoxy, optionally substituted C₃-C₈ cycloalkyl, optionally substituted C₆-C₁₀ bicycloalkyl, optionally substituted (C₁-C₄ alkyl)-aryl, optionally substituted aryl, optionally substituted (C₁-C₄ alkyl)-heterocycle, optionally substituted heterocycle, (C₁-C₄ alkyl)-OR¹⁰:

wherein the (C1-C4 alkyl) of the (C1-C4 alkyl)-OR10 is optionally substituted from 1 to 2 times with C1-C4 alkyl, optionally substituted aryl, optionally substituted heterocycle;

or R⁸ and R⁹, together with the nitrogen to which they are attached, combine to form an optionally substituted N-heterocycle;

R¹⁰ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, or optionally substituted heterocycle;

R¹¹ is independently at each occurrence optionally substituted C₁-C₆ alkyl, optionally substituted aryl, optionally substituted (C₁-C₄ alkyl)-aryl, optionally substituted (C₁-C₄ alkyl)-heterocycle, or optionally substituted heterocycle;

R¹² is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, optionally substituted heterocycle, or optionally substituted (C₁-C₄ alkyl)-heterocycle; or a pharmaceutical salt thereof.

R¹³ is independently at each occurrence C₁-C₆ alkyl or optionally substituted (C₁-C₄ alkyl)-aryl;

or a pharmaceutically acceptable salt thereof;

comprising the following steps:

(a) reacting a compound of formula II, wherein A and B are as defined above:

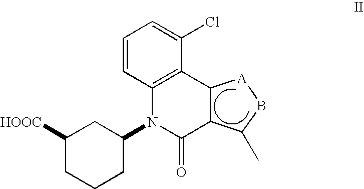

in an appropriate solvent, with an appropriate azide, then an appropriate alcohol, to provide a compound of formula (vi), wherein A and B are as defined above, and R′ is a lower alkyl group, benzyl, aryl, or heterocycle:

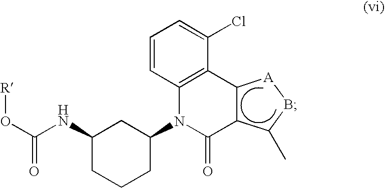

(b) deprotecting the compound of formula (vi), in an appropriate reaction medium, with an appropriate deprotecting agent to provide a compound of formula (vii), wherein A and B are as defined above:

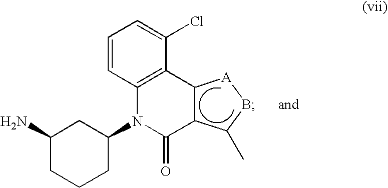

(c) acylating the compound of formula (vii), in a suitable solvent, with an appropriate acylating agent to provide a compound of formula I(a).

Furthermore, the invention relates to a process for converting the compounds of formulas II or m to form a compound of formula I(b), wherein R¹, A, and B are as defined above:

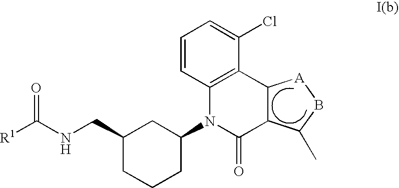

or a pharmaceutically acceptable salt thereof; comprising the following steps:

(a) reducing a compound of formula II, wherein A and B are as defined above:

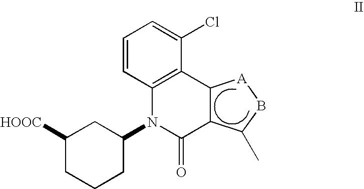

in an appropriate solvent, with an appropriate reducing agent to provide a compound of formula (x), wherein A and B are as defined above:

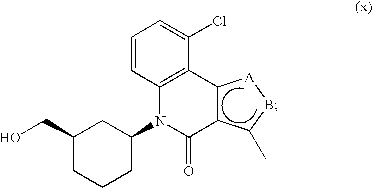

(b) reacting the compound of formula (x), in an appropriate solvent, with an appropriate nucleophile source to provide a compound of formula (xi), wherein A and B are as defined above and LG is a leaving group:

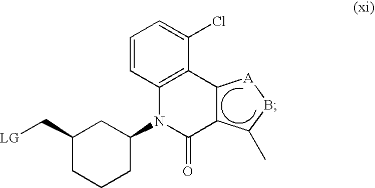

(c) reacting a compound of formula (xi), in an appropriate solvent, with an appropriate azide salt to provide a compound of formula (xii), wherein A and B are as defined above:

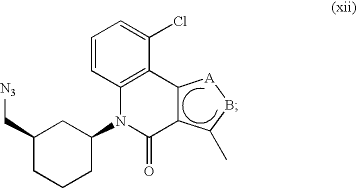

(d) reducing the compound of formula (xii), in an appropriate solvent, with an appropriate reducing agent to provide a compound of formula (xiv), wherein A and B are as defined above:

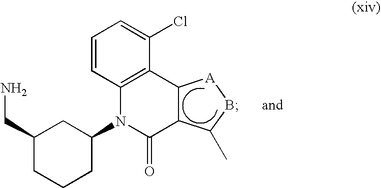

(e) acylating the compound of formula (xiv), in a suitable solvent, with an appropriate acylating agent to provide a compound of formula I(b).

Furthermore, the invention relates to a process for converting the compounds of formulas (i) to compounds of formula I(a), wherein R¹, A, and B are as defined above:

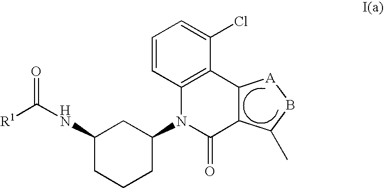

comprising the steps of:

(a) treating, in an appropriate solvent, a compound of formula (i),

wherein R is as defined above:

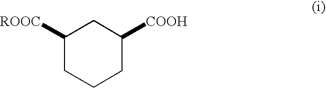

with an appropriate azide and subsequently an appropriate alcohol to form a compound of formula (ii), wherein R and R′ are defined above:

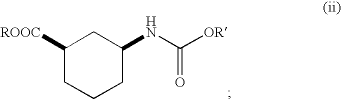

(b) deprotecting the compound of formula (ii), in an appropriate reaction medium, with an appropriate deprotecting agent to provide a compound of formula (iii), wherein R is as defined above:

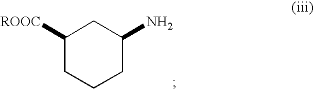

(c) reacting the compound of formula (iii), in an appropriate solvent, with (6chloro-2-fluorophenyl)-methylisoxazole-4carbonyl chloride to provide the compound of formula (iv), wherein R, A, and B are as defined above:

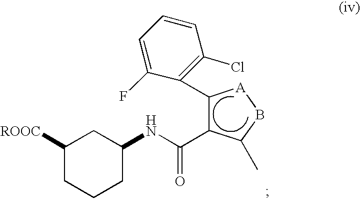

(d) cyclizing the compound of formula (iv), in an appropriate solvent, in the presence of an appropriate catalyst, to form a compound of formula III, wherein R, A, and B are as defined above:

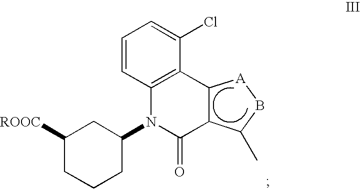

(e) hydrolyzing the compound of formula m to form a compound of formula III, wherein A and B are as defined above:

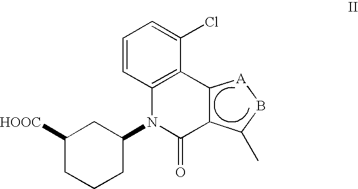

(f) reacting a compound of formula II in an appropriate solvent, with an appropriate azide, then an appropriate alcohol, to provide a compound of formula (vi), wherein R′, A, and B are as defined above:

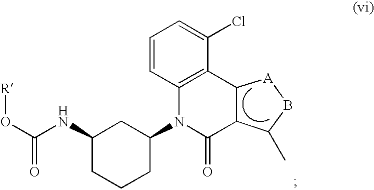

(g) deprotecting the compound of formula (vi), in an appropriate reaction medium, with an appropriate deprotecting agent to provide a compound of formula (vii), wherein A and B are as defined above:

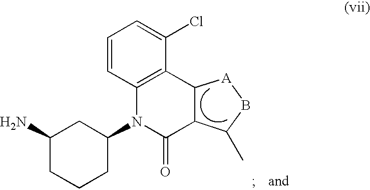

(h) acylating the compound of formula (vii), in a suitable solvent, with an appropriate acylating agent to provide a compound of formula I(a).

Furthermore, the invention relates to a process for converting the compounds of formulas (i) to compounds of formula I(b), wherein R¹, A, and B are as defined above:

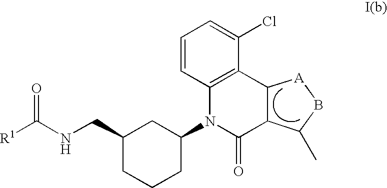

or a pharmaceutically acceptable salt thereof;

comprising the steps of:

(a) treating, in an appropriate solvent, a compound of formula (i), wherein R is as defined above:

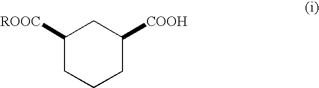

with an appropriate azide and subsequently an appropriate alcohol to form a compound of formula (ii), wherein R is defined above and R' is a lower alkyl group, benzyl, aryl, or heterocycle:

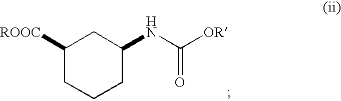

(b) deprotecting the compound of formula (ii), in an appropriate reaction medium, with an appropriate deprotecting agent to provide a compound of formula (iii), wherein R is as defined above:

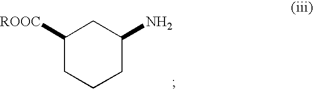

(c) reacting the compound of formula (iii), in an appropriate solvent, with (6-chloro-2-fluorophenyl)-methylisoxazole-4-carbonyl chloride to provide the compound of formula (iv), wherein R, A, and B are as defined above:

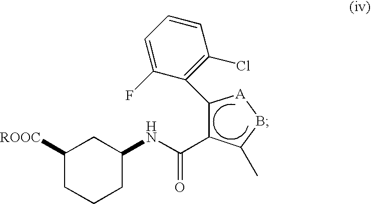

(d) cyclizing the compound of formula (iv), in an appropriate solvent, in the presence of an appropriate catalyst, to form a compound of formula III, wherein R, A, and B are as defined above:

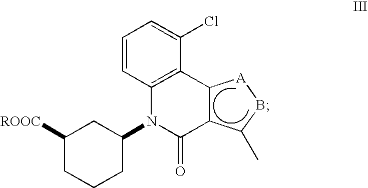

(e) hydrolyzing the compound of formula III to form a compound of formula II, wherein A and B are as defined above:

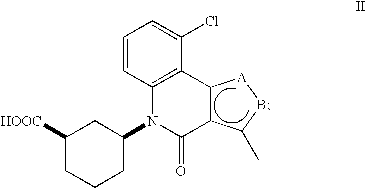

(f) reducing a compound of formula II in an appropriate solvent, with an appropriate reducing agent to provide a compound of formula (x), wherein A and B are as defined above:

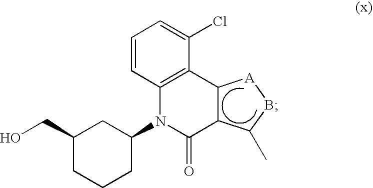

(g) reacting the compound of formula (x), in an appropriate solvent, with an appropriate nucleophile source to provide a compound of formula (xi), wherein A and B are as defined above and LG is leaving group:

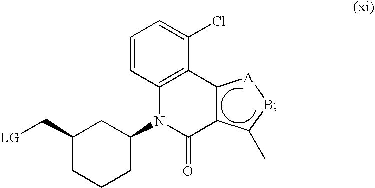

(h) reacting a compound of formula (xi), in an appropriate solvent, with an appropriate azide salt to provide a compound of formula (xii), wherein A and B are as defined above:

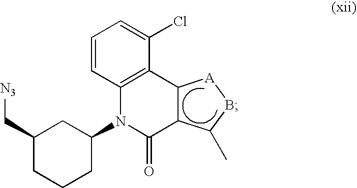

(i) reducing the compound of formula (xii), in an appropriate solvent, with an appropriate reducing agent to provide a compound of formula (xiv), wherein A and B are as defined above:

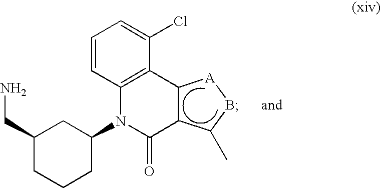

(j) acylating the compound of formula (xiv), in a suitable solvent, with an appropriate acylating agent to provide a compound of formula I(b).

Definitions and General Parameters

The following definitions are set forth to illustrate and define the meaning and scope of the various terms used to describe the invention herein.

In general, the term “pharmaceutical” when used as an adjective means substantially non-toxic to living organisms. For example, the term “pharmaceutical salt” as used herein, refers to salts of the compounds of formula I which are substantially non-toxic to living organisms. See, e.g., Berge, S. M, Bighley, L. D., and Monkhouse, D. C., “Pharmaceutical Salts”, J. Phann. Sci., 66:1, 1977. Typical pharmaceutical salts include those salts prepared by reaction of the compounds of formula I with an inorganic or organic acid or base. Such salts are known as acid addition or base addition salts respectively. These pharmaceutical salts frequently have enhanced solubility characteristics compared to the compound from which they are derived, and thus are often more amenable to formulation as liquids or emulsions.

The term “acid addition salt” refers to a salt of a compound of formula I prepared by reaction of a compound of formula I with a mineral or organic acid. For exemplification of pharmaceutical acid addition salts see, e.g., Berge, S. M, Bighley, L. D., and Monkhouse, D. C., J. Pharm. Sci., 66:1, 1977. Since compounds of this invention can be basic in nature, they accordingly react with any of a number of inorganic and organic acids to form pharmaceutical acid addition salts.

The pharmaceutical acid addition salts of the invention are typically formed by reacting the compound of formula I with an equimolar or excess amount of acid. The reactants are generally combined in a mutual solvent such as diethylether, tetrahydrofuran, methanol, ethanol, isopropanol, benzene, and the like. The salts normally precipitate out of solution within about one hour to about ten days and can be isolated by filtration or other conventional methods.

Acids commonly employed to form acid addition salts are inorganic acids such as hydrochloric acid, hydrobromic acid, hydroiodic acid, sulfuric acid, phosphoric acid, and the like, and acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, 1,5-naphthalene-disulfonate, naphthalene-1-sulfonate, naphthalene-2-sulfonate, mandelate and the like.

The term “base addition salt” refers to a salt of a compound of formula I prepared by reaction of a compound of formula I with a mineral or organic base. For exemplification of pharmaceutical base addition salts see, e.g., Berge, S. M, Bighley, L. D., and Monkhouse, D. C., J. Pharm. Sci., 66:1, 1977. This invention also contemplates pharmaceutical base addition salts of compounds of formula I. The skilled artisan would appreciate that some compounds of formula I may be acidic in nature and accordingly react with any of a number of inorganic and organic bases to form pharmaceutical base addition salts. Examples of pharmaceutical base addition salts are the ammonium, lithium, potassium, sodium, calcium, magnesium, methylamino, diethylamino, ethylene diamino, cyclohexylamino, and ethanolamino salts, and the like of a compound of formula I.

The terms “inhibit” as it relates to MRP1 and “inhibiting MRP1” refer to prohibiting, alleviating, ameliorating, halting, restraining, slowing or reversing the progression of, or reducing MRP1's ability to redistribute an oncolytic away from the oncolytic's site of action, most often the neoplasm's nucleus, and out of the cell.

The term “effective amount of a compound of formula I” refers to an amount of a compound of the present invention which is capable of inhibiting MRP1. The term “effective amount of an oncolytic agent” refers to an amount of oncolytic agent capable of inhibiting a neoplasm, resistant or otherwise.

The term “inhibiting a resistant neoplasm, or a neoplasm susceptible to resistance” refers to prohibiting, halting, restraining, slowing or reversing the progression of, reducing the growth of, or killing resistant neoplasms and/or neoplasms susceptible to resistance.

The term “resistant neoplasm” refers to a neoplasm, which is resistant to chemotherapy where that resistance is conferred in part, or in total, by MRP1. Such neoplasms include, but are not limited to, neoplasms of the bladder, bone, breast, lung(small-cell), testis, and thyroid and also includes more particular types of cancer such as, but not limited to, acute lymphoblastic and myeloblastic leukemia, Wilm's tumor, neuroblastoma, soft tissue sarcoma, Hodgkin's and non-Hodgkin's lymphomas, and bronchogenic carcinoma.

A neoplasm, which is “susceptible to resistance”, is a neoplasm where resistance is not inherent nor currently present but can be conferred by MRP1 after chemotherapy begins. Thus, the methods of this invention encompass a prophylactic and therapeutic administration of a compound of formula I.

The term “chemotherapy” refers to the use of one or more oncolytic agents where at least one oncolytic agent is a substrate of MRP1. A “substrate of MRP1” is an oncolytic that binds to MRP1 and is redistributed away from the oncolytic's site of action (the nucleus of the neoplasm) and out of the cell, thus, rendering the therapy less effective. Preferred oncolytic agents are doxorubicin, daunorubicin, epirubicin, vincristine, and etoposide.

The terms “treat” or “treating” bear their usual meaning which includes preventing, prohibiting, alleviating, ameliorating, halting, restraining, slowing or reversing the progression, or reducing the severity of MRP1 derived drug resistance in a multidrug resistant tumor.

In the general formulae of the present document, the general chemical terms have their usual meanings. For example, the term “C₁-C₄ alkyl” refers to methyl, ethyl, propyl, isopropyl, cyclopropyl, butyl, cyclobutyl, s-butyl, and t-butyl. The term “C₁-C₆ alkyl” refers to a monovalent, straight or branched saturated hydrocarbon containing from 1 to 6 carbon atoms. Additionally, the term “C₁-C₆ alkyl” includes C₁-C₄ alkyl groups and C₃-C₆ cycloalkyls. The term “C₁-C₆ alkyl” includes, but is not limited to, cyclopentyl, pentyl, hexyl, cyclohexyl, and the like. The term “C₃-C₈ cycloalkyl” refers to cyclopropyl, cyclobutyl, cyclopentyl, cyclohexyl, cycloheptyl, and cyclooctyl. The term “C₅-C₇ cycloalkyl” refers to cyclopentyl, cyclohexyl, and cycloheptyl. The term “C₆-C₁₀ bicycloalkyl” refers to bicyclo-[2.1.1]hexanyl, [2.2.1]heptanyl, [3.2.1]octanyl, [2.2.2]octanyl, [3.2.2]nonanyl, [3.3.1]nonanyl, [3.3.2]decanyl, and [4.3.1]decanyl ring systems.

The term “lower alkyl” refers to branched or straight chain monovalent alkyl radical of one to six carbon atoms, and optionally to a cyclic monovalent alkyl radical of three to six carbon atoms. This term is further exemplified by such radicals as methyl, ethyl, n-propyl, isopropyl, n-butyl, t-butyl, i-butyl (or 2-methylpropyl), cyclopropyl-methyl, i-amyl, n-amyl, and hexyl.

The terms “optionally substituted C₁-C₄ alkyl” and “optionally substituted C₁-C₆ alkyl” refers to a C₁-C₄ alkyl or C₁-C₆ alkyl, respectively, unsubstituted or substituted from 1 to 3 times with halo or hydroxy.

The terms “C₁-C₄ alkoxy” and “C₁-C₆ alkoxy” refer to moieties of the formula O-(C₁-C₄ alkyl) and O-(C₁-C₆ alkyl) respectively.

The term “optionally substituted C₃-C₈ cycloalkyl” refers to a C₃-C₈ cycloalkyl unsubstituted or substituted once with a phenyl, substituted phenyl, or CO₂R² group.

The terms “optionally substituted (C₁-C₄ alkyl)-(C₃-C₈ cycloalkyl)” refers to optionally substituted C₃-C₈ cycloalkyl linked through a C₁-C₄ alkyl, unsubstituted or substituted with halo or hydroxy.

The term “optionally substituted O-(C₃-C₈ cycloalkyl)” refers to an optionally substituted C₃-C₈ cycloalkyl linked through an oxygen atom.

The term “optionally substituted C₆-C₁₀ bicycloalkyl” refers to a C₆-C₁₀ bicycloalkyl unsubstituted or substituted once with a phenyl, substituted phenyl, or CO₂R² group.

The term “halo” or “halide” refers to fluoro, chloro, bromo, and iodo.

The term “aryl” refers to phenyl, and naphthyl.

The terms “optionally substituted aryl” refers to a phenyl and naphthyl group, respectively, unsubstituted or substituted from 1 to 5 times independently with C₁-C₆ alkyl, C₁-C₄ alkoxy, halo, hydroxy, trifluoromethyl, NR⁸R⁹, SO₂N(R¹⁰)₂, NH-Pg, C₁-C₆ alkoxy, benzyloxy, C(O)R¹⁰, C₅-C₇ cycloalkyl, trifluoromethoxy, SR², cyano, or nitro.

The terms “optionally substituted (C₁-C₄ alkyl)-aryl” refers to optionally substituted aryl linked through a C₁-C₄ alky, unsubstituted or substituted with halo, trifluoromethyl, or hydroxy.

The term “optionally substituted O-aryl” refers to an optionally substituted aryl linked through an oxygen atom.

The terms “optionally substituted phenoxy” refers to a phenoxy group unsubstituted or substituted from 1 to 3 times independently with C₁-C₆ alkyl, halo, hydroxy, trifluoromethyl, NR⁸R⁹, SO₂N(R¹⁰)₂, NH-Pg, C₁-C₆ alkoxy, benzyloxy, C(O)R¹⁰, C₅-C₇ cycloalkyl, trifluoromethoxy, or nitro.

The terms “optionally substituted (C₁-C₄ alkyl)-phenoxy” refers to unsubstituted or substituted phenoxy linked through a C₁-C₄ alkyl, optionally substituted with halo, trifluoromethyl, or hydroxy.

The term “heterocycle” is taken to mean stable unsaturated and saturated 3 to 6 membered rings containing from 1 to 4 heteroatoms selected from the group consisting of nitrogen, oxygen and sulfur, said rings being optionally benzofused. All of these rings may be substituted with up to three substituents independently selected from the group consisting of halo, C₁-C₄ alkoxy, C₁-C₄ alkyl, cyano, nitro, hydroxy, —S(O)_{d}-(C₁-C₄ alkyl) and —S(O)_{d}-phenyl where d is 0, 1 or 2. Saturated rings include, for example, pyrrolidinyl, piperidinyl, piperazinyl, tetrahydrofuryl, oxazolidinyl, morpholino, dioxanyl, pyranyl, and the like. Benzofused saturated rings include indolinyl, 1,2,3,4-tetrahydroquinolinyl, 1,2,3,4-tetrahydroisoquinolinyl and the like. Unsaturated rings include furyl, thienyl, pyridinyl, pyrrolyl, N-methylpyrrolyl, oxazolyl, isoxazolyl, pyrazolyl, imidazolyl, triazolyl, oxadiazolyl, thiadiazolyl, thiazolyl, pyrimidinyl, pyrazinyl, pyridazinyl, and the like.

Benzofused unsaturated rings include isoquinolinyl, benzoxazolyl, benzthiazolyl, quinolinyl, benzofuranyl, thionaphthyl, indolyl and the like.

The term “heteroaryl” is taken to mean an unsaturated or benzofused unsaturated heterocycle as defined in the previous paragraph.

The term “optionally substituted heterocycle” refers to a heterocyclic ring unsubstituted or substituted 1 or 3 times independently with a C₁-C₆ alkyl, halo, benzyl, phenyl, trifluoromethyl. Heterocyclic rings may be additionally substituted 1 or 2 times with an oxo group, however, total substitution of the saturated heterocyclic ring may not exceed two substituents.

The term “optionally substituted O-heterocycle” refers to an optionally substituted heterocycle linked through an oxygen atom.

The terms “optionally substituted (C₁-C₄ alkyl)-heterocycle” refers to optionally substituted heterocycle linked through a C₁-C₄ alkyl, unsubstituted or substituted with halo or hydroxy.

The term “N-heterocycle” refers to a nitrogen containing heterocycle linked through nitrogen atom.

The term “amino acid ester” as used in this specification refers to an amino acid where the carboxy group is substituted with a C₁-C₆ alkyl or benzyl group. That is, the alkyl group when taken together with the carboxy group forms a C₁-C₆ alkyl ester. A skilled artisan would appreciate that some amino acids have two carboxy groups that may be substituted with a C₁-C₆ alkyl group, for example, aspartic acid and glutamic acid.

This invention contemplates the possibility of amino acid mono- or diesters in these circumstances.

The term “protecting group” (Pg) refers to an amino protecting group or a hydroxy protecting group. The species of protecting group will be evident from whether the “Tg” group is attached to a nitrogen atom (amino protecting group) or attached to an oxygen atom (hydroxy protecting group).

The term “leaving group” as used herein refers to a group cleavable from the substrate molecule during a reaction step and comprises a halo group, sulfonates (e.g., mesylate (OMs) or tosylate (OTs)) and the like known in the art as leaving groups.

The term “nucleophile source” as used herein describes a group capable of effecting a nucleophilic substitution on an alcohol. Such groups include halogenic acids such as HCl, HBr or IR and sulfonic acids, sulfonic anhydrides or sulfonic acid halides e.g., methanesulfonic acid chloride.

The term “amino protecting group” as used in this specification refers to a substituent(s) of the amino group commonly employed to block or protect the amino functionality while reacting other functional groups on the compound. Examples of such amino-protecting groups include the formyl group, the trityl group, the phthalimido group, the acetyl group, the trichloroacetyl group, the chloroacetyl, bromoacetyl, and iodoacetyl groups, urethane-type blocking groups such as benzyloxycarbonyl, 9-fluorenylmethoxycarbonyl (“BLOC”), and the like; and like amino protecting groups. The species of amino protecting group employed is not critical so long as the derivatized amino group is stable to the condition of subsequent reaction(s) on other positions of the molecule and can be removed at the appropriate point without disrupting the remainder of the molecule. Similar amino protecting groups used in the cephalosporin, penicillin, and peptide arts are also embraced by the above terms. Further examples of groups referred to by the above terms are described by T. W. Greene, “Protective Groups in Organic Synthesis”, John Wiley and Sons, New York, N.Y., 1991, Chapter 7 hereafter referred to as “Greene”. A preferred amino protecting group is t-butyloxycarbonyl.

The term “hydroxy protecting group” denotes a group understood by one skilled in the organic chemical arts of the type described in Chapter 2 of Greene. Representative hydroxy protecting groups include, for example, ether groups including methyl and substituted methyl ether groups such as methyl ether, methoxymethyl ether, methylthiomethyl ether, tert-buylthiomethyl ether, (phenyldimethylsilyl)methoxy-methyl ether, benzyloxymethyl ether, p-methoxybenzyloxy-methyl ether, and tert-butoxymethyl ether; substituted ethyl ether groups such as ethoxyethyl ether, 1-(2-chloroethoxy)-ethyl ether, 2,2,2-trichloroethoxymethyl ether, and 2-(trimethylsilyl)ethyl ether; isopropyl ether groups; phenyl and substituted phenyl ether groups such as phenyl ether, p-chlorophenyl ether, p-methoxyphenyl ether, and 2,4-dinitrophenyl ether; benzyl and substituted benzyl ether groups such as benzyl ether, p-methoxybenzyl ether, o-nitrobenzyl ether, and 2,6-dichlorobenzyl ether; and alkylsilyl ether groups such as trimethyl- triethyl- and triisopropylsilyl ethers, mixed alkylsilyl ether groups such as dimethylisopropylsilyl ether, and diethylisopropylsilyl ether; and ester protecting groups such as formate ester, benzylformate ester, mono-, di-, and trichloroacetate esters, phenoxyacetate ester, and p-chlorophenoxyacetate and the like. The species of hydroxy protecting group employed is not critical so long as the derivatized hydroxy group is stable to the conditions of subsequent reaction(s) on other positions of the intermediate molecule and can be selectively removed at the appropriate point without disrupting the remainder of the molecule including any other hydroxy protecting group(s).

The term “MTBE” as used herein describes methyl tertiary-butyl ether. The term “KHMDS” as used herein describes potassium hexamethyldisilazane.

Compounds of formula I, wherein A, B, and R¹ are as defined previously and n is 0 or 1:

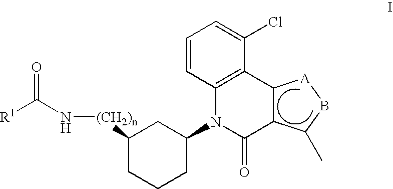

or a pharmaceutically acceptable salt thereof; are selective P1 inhibitors, which may be prepared from the compounds of formula II:

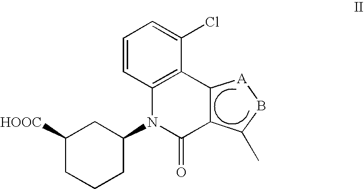

by methods well known in the art, see for example Scheme 1 and Scheme 2.

### Scheme 1

Preparation of a Compound of Formula I(a), Wherein R′, R¹, A, and B are as Previously Described.

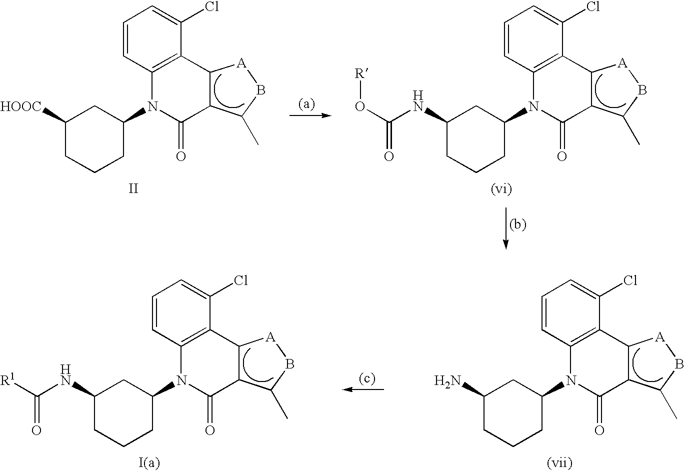

Compounds of formula I(a) may be prepared by a process comprising the following steps:

(a) reacting a compound of formula II in an appropriate solvent, with an appropriate azide, then an appropriate alcohol, to provide a compound of formula (vi):

(b) deprotecting the compound of formula (vi), in an appropriate reaction medium, with an appropriate deprotecting agent to provide a compound of formula (vii):

(c) acylating the compound of formula (vii), in a suitable solvent, with an appropriate acylating agent to provide a compound of formula I(a).

For examples of how to perform such reactions see Org. Rxs 3 337 (1947), TL 25 3515 (1984), JOC 51 3007, 5123 (1986); 52 4875 (1987), JOC 26 3511 (1961); 43 2164 (1978), JACS 94 6203 (1972), Larock 431-432, and Tetr 30 2151 (1974).

Step (a)

The process of step (a) of the invention is performed via the Curtius rearrangement, by converting a compound of formula II, in an appropriate solvent, with an appropriate azide, then an appropriate alcohol, to provide a compound of formula (vi). As the skilled artisan would appreciate, the compound of formula II, dissolved in an appropriate solvent, is first treated with an appropriate azide and optionally a catalyst to provide the intermediate. The intermediate is treated with an appropriate alcohol to obtain the compound of formula (vi). Once the reaction is complete, as measured by the consumption of the substrate, the resulting compound of formula (vi) may be isolated by standard extractions and filtrations. If desired, the resulting compound of formula (vi) may be further purified by chromatography or crystallization as appropriate.

Appropriate solvents must be capable of dissolving a sufficient amount of the compound of formula II and the azide for the reaction to proceed. Useful organic solvents include hexamethylphosphoramide, dimethylformamide, and preferably toluene.

The skilled artisan would appreciate that the Curtius rearrangement may be performed via a number of azides and that reaction conditions may vary depending upon the azide used. For example if sodium azide, potassium azide, and the like are used the compound must first be converted to the activated acid with an appropriate activating agent, such as ethyl chloroformate or sulfuric acid. The substrate may need to be pretreated with the activating agent, such as the case with ethyl chloroformate, or may need to be added simultaneously. The skilled artisan would appreciate the potential for reaction at the ester site of the substrate, if the molecule is treated with the azide first as is the case in these circumstances.

Preferably, diphenylphosphoryl azide is used in the process of the present invention without an activating agent.

Appropriate alcohols for step (a) of the invention are lower alkyl alcohols such as methanol, ethanol, propanol, isopropanol, butanol, benzyl, t-butanol, TMS-ethanol, and the like.

The process of step (a) may be carried out over a large range of concentrations, from about 0.001 molar to about 2.0 molar of the azide, dependent upon the solubility of the particular azide in the chosen solvent. The reaction may also be performed on slurries of the azide so long as a sufficient amount of the azide is soluble in the solvent for the reaction to proceed. Preferably the process is performed at a concentration from about 0.1 molar to about 1.0 molar. A concentration of about 0.3 to about 0.4 molar is most preferred.

Reactions of step (a) may be performed between about 80° C. and about 130° C., preferably between about 100° C. and about 120° C. Most preferably the reactants are combined at temperature of about 20° C. to about 30° C., then heated to about 80° C. and about 120° C., the azide is then added, and the reactants are stirred for about 0.5 to about 1.5 hours at reflux. An appropriate alcohol is then added and heated to about 70° C. to about 90° C. for about 3 to about 24 hours, preferably from about 75° C. to about 85° C. for about 8 to about 12 hours.

Step (b)

The deprotection process of step (b) of the invention is performed by combining the compound of formula (vi) with an appropriate deprotecting agent in an appropriate reaction medium. Once the reaction is complete, as measured by consumption of the substrate, the resulting compound of formula (vii) is isolated by standard extractions and filtrations. If desired, the resulting compound of formula (vii) may be further purified by chromatography or crystallization as appropriate. Methods of removing an amino-protecting group are well known in the art, for example, see T. W. Greene, “Protective Groups in Organic Synthesis”, John Wiley and Sons, New. York, N.Y., 1991, Chapter 7 hereafter referred to as “Greene”.

Compounds useful as deprotecting agents, those compounds which cleave the protecting group from the compound of formula (vi), depend on the protecting group used, as the skilled artisan would appreciate. For example, a strong acid, such as trifluoroacetic acid or iodotrimethylsilane will remove a BOC protecting group.

Reaction media useful for the process of step (b) must be capable of dissolving a sufficient amount of the compound of formula (vi) for the reaction to proceed. Organic solvents useful as reaction media for the process of this invention depend upon the choice of deprotecting agent and may include tetrahydrofuran, acetonitrile or chlorocarbons.

Depending upon the choice of deprotecting agent, reactions of step (b) may be performed between about −30° and about 60° C. The skilled artisan will appreciate that the reaction rates will decrease as temperatures are lowered and increase as temperatures are elevated.

The process of step (b) may be carried out over a large range of concentrations, from about 0.001 molar to about 2.0 molar of the compound of formula (vi), dependent upon the solubility of the particular product in the chosen reaction medium. Preferably the process is performed at a concentration from about 0.01 molar to about 1.0 molar. A concentration of about 0.20 molar to about 0.50 molar is most preferred.

Step (c)

The acylation process of step (c) of the invention is performed by combining the compound of formula (vii) with an appropriate acylating agent in an appropriate solvent. Once the reaction is complete, as measured by consumption of the substrate, the resulting compound of formula I(a) is isolated by standard extractions and filtrations. If desired, the resulting compound of formula I(a) may be further purified by chromatography or crystallization as appropriate.

Reductive alkylation of primary amines are well known transformations, see, e.g., Larock, pg. 434-435. The skilled artisan will appreciate that the treatment of acyl halides, preferably the acyl chlorides, with amines is a very general reaction for the preparation of amides, see, e.g. March J, Advanced Organic Chemistry, 1985, 3rd edition, page 370. The reaction is highly exothermic and must be carefully controlled, usually by cooling.

Reaction media useful must be capable of dissolving a sufficient amount of the compound of formula (vii) for the reaction to proceed. Organic solvents useful as reaction media for the process of this invention depend upon the choice of alkylating agent and may include CHCl₃, hexane, cyclohexane, nitromethane, nitrobenzene, acetonitrile, ether, dioxane, trichloroacetic anhydride, dichloroacetic anhydride, and preferably tetrahydrofuran or CH₂Cl₂.

The overall conversion may be performed at about 0° C. to the boiling point of the mixture but room temperature is a preferred reaction temperature. The formation of the compounds of formulas I(a) may take from 15 minutes to 24 hours as measured by the consumption of the acyl halide.

The acyl halides of the present invention are commercially available, and to the extent not available, may be prepared by methods well known to the skilled artisan.

### Scheme 2

### Preparation of a Compound of Formula I(b)

Preparation of a Compound of Formula I(b), Wherein R, R¹, A, and B are as Previously Described.

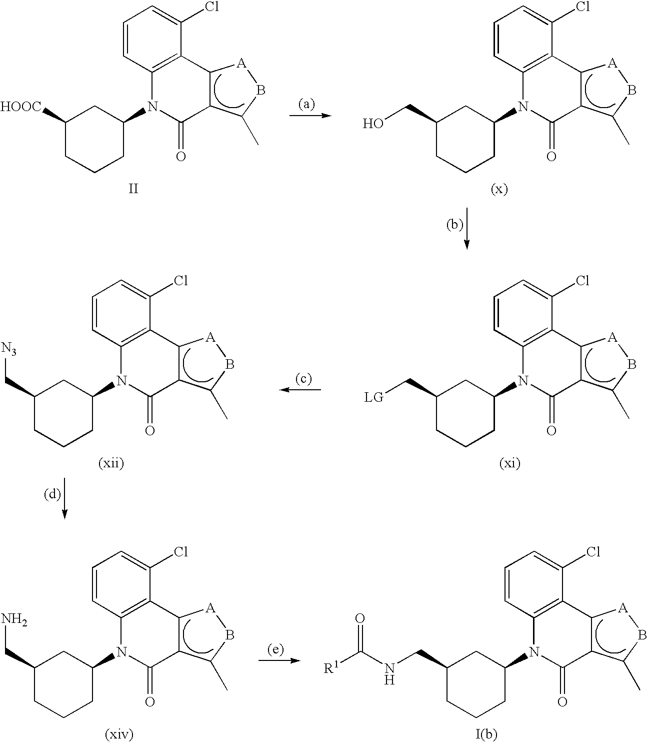

Compounds of formula I(b) may be prepared by a process comprising the following steps:

(a) reducing a compound of formula II in an appropriate solvent, with an appropriate reducing agent to provide a compound of formula (x);

(b) reacting the compound of formula (x), in an appropriate solvent, with an appropriate nucleophile source to provide a compound of formula (xi);

(c) reacting a compound of formula (xi), in an appropriate solvent, with an appropriate azide salt to provide a compound of formula (xii);

(d) reducing the compound of formula (xii), in an appropriate solvent, with an appropriate reducing agent to provide a compound of formula (xiv); and

(e) acylating the compound of formula (xiv), in a suitable solvent, with an appropriate acylating agent to provide a compound of formula I(b).

Step (a)

The process of step (a) of the invention is performed by reducing a compound of formula II, in an appropriate solvent, with an appropriate reducing agent to provide a compound of formula (x). Once the reaction is complete, as measured by the consumption of the substrate, the resulting compound of formula (x) may be isolated by standard extractions and filtrations. If desired, the resulting compound of formula (x) may be further purified by chromatography or crystallization as appropriate.

The carboxylic acid of formula II may be reduced to the corresponding alcohol by methods well known in the art, see for example Larock, pages 548-549. This reaction proceeds by combining the carboxylic acid with an appropriate reducing agent in an appropriate solvent.

Appropriate reducing agents include boron compounds such as BH₃, BH₃.SMe₂, BF₃.OEt₂, B(OMe)₃, LiBH₄, and NaBH₄. Appropriate solvents useful when the reducing agent is a boron compound must be capable of dissolving a sufficient amount of the carboxylic acid for the reaction to proceed. Such solvents may include the following organic solvents: dichloromethane, mixtures of DMF and dichloromethane, mixtures of tetrahydrofuran and water, dimethylformamide, and preferably tetrahydrofuran. Additional appropriate reducing agents include aluminum compounds such as LiAlH_{3 and NaH}₂Al(OCH₂CH₂OCH₃)₂, and other reducing agents that reduce the carboxylic acid without adversely affecting the rest of the molecule. The skilled artisan would appreciate that the solvents useful for aluminum compounds are well known in the art and include such solvents as dichloromethane, benzene, diethylamine, tetrahydrofuran, and the like.

The skilled artisan would appreciate that the reducing agent is preferably added dropwise to the substrate at about −5° C. to about 10° C. then slowly warmed to room temperature. The solution is stirred for about ½ to about 2 hours, then quenched.

Once the reaction is complete, as measured by the consumption of the acid, the resulting alcohol may be isolated by standard extractions and filtrations. If desired, the alcohol may be further purified by chromatography or crystallization as appropriate.

Step (b)

The process of step (b) of the invention is performed by reacting the compound of formula (x), in an appropriate solvent, with an appropriate nucleophile source to provide a compound of formula (xi).

Appropriate solvents must be capable of dissolving a sufficient amount of the compound of formula (x) for the reaction to proceed. The skilled artisan would appreciate that the solvent selected depends upon the nucleophile source used. Useful organic solvents include dichloromethane, TBF, CHCl₃, dioxane, benzene, MeCN, HCONMe₂, Ac₂O, acetone, ethanol, and preferably pyridine.

Appropriate sources of nucleophiles include HCl, HBr or HI and sulfonic acids, sulfonic anhydrides or sulfonic acid halides e.g., methanesulfonic acid chloride or the like. Bromination, for example, is effected by the addition of hydrogen bromide while maintaining a temperature from about 30° C. to about 100° C., for about 2 to about 5 hours, preferably about 4 hours. The reaction mixture is then evaporated to dryness, affording the corresponding compound. The use of methanesulfonyl chloride is preferred.

Step (c)

The process of step (c) of the invention is performed via nucleophilic displacement, by reacting a compound of formula (xi), in an appropriate solvent, with an appropriate azide salt to provide a compound of formula (xii). Such reactions are well known in the art, see for example Chemistry Letters 635 (1977). As the skilled artisan would appreciate, the compound of formula (xi), dissolved in an appropriate solvent, is treated with an appropriate azide to provide a compound of formula (xii). Once the reaction is complete, as measured by the consumption of the substrate, the resulting compound of formula (xii) may be isolated by standard extractions and filtrations. If desired, the resulting compound of formula (xii) may be further purified by chromatography or crystallization as appropriate.

Appropriate solvents must be capable of dissolving a sufficient amount of the compound of formula (xi) and the azide for the reaction to proceed. Useful organic solvents include hexamethylphosphoramide, toluene, and preferably dimethylformamide.

Suitable azides for this reaction include sodium azide, potassium azide, and the like. Preferably sodium azide is the azide of choice

The process of step (c) may be carried out over a large range of concentrations, from about 0.001 molar to about 4.0 molar of the azide, dependent upon the solubility of the particular azide in the chosen solvent. The reaction may also be performed on slurries of the azide so long as a sufficient amount of the azide is soluble in the solvent for the reaction to proceed. Preferably the process is performed at a concentration from about 2.0 molar to about 4.0 molar. A concentration of about 3.0 to about 3.5 molar is most preferred.

Reactions of step (c) may be performed between about 20° C. and about 130° C., preferably between about 50° C. and about 70° C. Most preferably the reactants are combined at temperature of about 20° C. to about 30° C., then heated to a temperature of about 50° C. to about 70° C. and stirred for about 1.0 to about 20.0 hours.

Step (d)

The reduction of step (d) of the invention is performed by combining the compound of formula (xii) with an appropriate reducing agent in an appropriate reaction medium. Once the reaction is complete, as measured by consumption of the substrate, the resulting compound of formula (xiv) is isolated by standard extractions and filtrations. If desired, the resulting compound of formula (xiv) may be further purified by chromatography or crystallization as appropriate. Methods of reducing an azide to an amine are well known in the art, for example, see Larock, (2^{nd} ed., 1999), pages 815-820.

Compounds useful as reducing agents include NaBH₄, LiBH₄, triphenyl phosphine, and the like. Additionally the reducing agent may be H₂ in the presence of a suitable catalyst, such as Pd/C, Pd, Raney Ni, Pd—Al₂O₃, Pd(OH)₂—C, PdO, PtO₂, and the like.

Reaction media useful for the process of step (e) must be capable of dissolving a sufficient amount of the compound of formula (xii) for the reaction to proceed. Organic solvents useful as reaction media for the process of this invention depend upon the choice of reducing agent and may include ethyl acetate and ethanol.

Depending upon the choice of reducing agent, reactions of step (d) may be performed between about −30° and about 60° C. Preferable the reaction is performed between about 20° C. and about 30° C. The skilled artisan will appreciate that the reaction rates will decrease as temperatures are lowered and increase as temperatures are elevated.

Step (e)

The acylation process of step (e) of the invention is performed by combining the compound of formula (xiv) with an appropriate acylating agent in an appropriate solvent. Once the reaction is complete, as measured by consumption of the substrate, the resulting compound of formula I(b) is isolated by standard extractions and filtrations. If desired, the resulting compound of formula I(b) may be further purified by chromatography or crystallization as appropriate.

Acylations of primary amines are well known transformations, see, e.g., Larock, pg. 434-435. The skilled artisan will appreciate that the treatment of acyl halides, preferably the acyl chlorides, with amines is a very general reaction for the preparation of amides, see, e.g. March J, Advanced Organic Chemistry, 1985, 3rd edition, page 370. The reaction is highly exothermic and must be carefully controlled, usually by cooling.

Reaction media useful must be capable of dissolving a sufficient amount of the compound of formula (xiv) for the reaction to proceed. Organic solvents useful as reaction media for the process of this invention depend upon the choice of acylating agent and may include CHCl₃, hexane, cyclohexane, nitromethane, nitrobenzene, acetonitrile, ether, dioxane, trichloroacetic anhydride, dichloroacetic anhydride, and preferably tetrahydrofuran or CH₂Cl₂.

The overall conversion may be performed at about 0° C. to the boiling point of the mixture but room temperature is a preferred reaction temperature. The formation of the compounds of formulas I(b) may take from 15 minutes to 24 hours as measured by the consumption of the acyl halide.

The acyl halides of the present invention are commercially available, and to the extent not available, may be prepared by methods well known to the skilled artisan.

Compounds of formula II, wherein R may be lower alkyl, benzyl, heterocycle, and aryl may be prepared by the process of this invention as defined by Scheme 3:

### Scheme 3

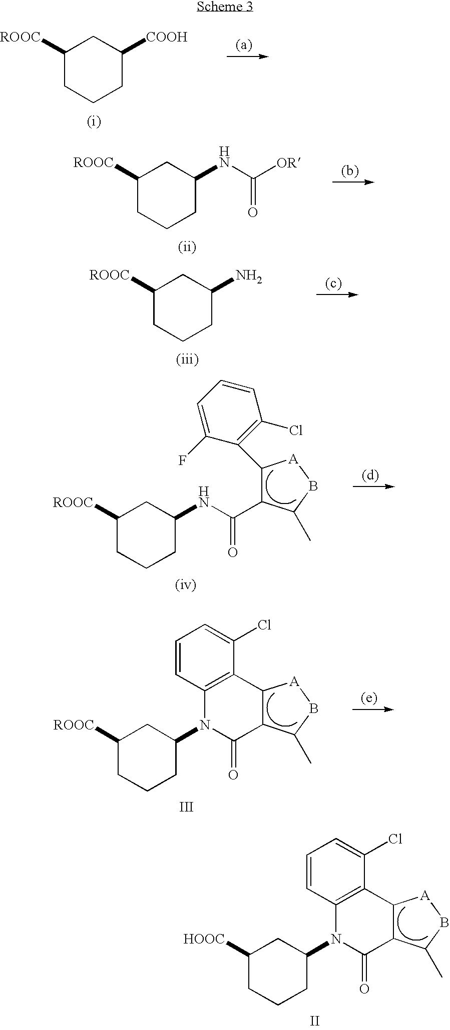

comprising:

(a) treating, in an appropriate solvent, a compound of formula (i), with an appropriate azide and subsequently an appropriate alcohol to form a compound of formula (ii);

(b) deprotecting the compound of formula (ii), in an appropriate reaction medium, with an appropriate deprotecting agent to provide a compound of formula (iii);

(c) reacting the compound of formula (iii), in an appropriate solvent, with (6-chloro-2-fluorophenyl)-methylisoxazole-4-carbonyl chloride to provide the compound of formula (iv);

(d) cyclizing the compound of formula (iv), in an appropriate solvent, in the presence of an appropriate catalyst, to form a compound of formula III; and

(e) optionally, hydrolyzing the compound of formula m to form a compound of formula II.

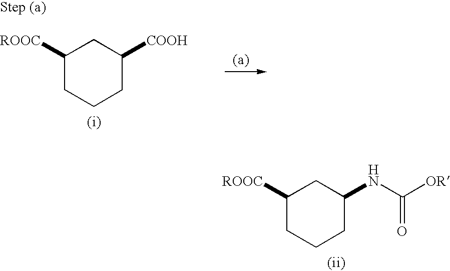

The process of step (a) of the invention is performed via Curtius rearrangement by (1) converting the compound of formula (i) to the corresponding acyl azide and then (2) treating the corresponding acyl azide to form the compound of formula (ii) with an appropriate substrate. For examples of how to perform such reactions see Org. Rxs 3 337 (1947), TL 25 3515 (1984), JOC 51 3007, 5123 (1986); 52 4875 (1987), JOC 26 3511 (1961); 43 2164 (1978), and preferably JACS 94 6203 (1972) and Tetr 30 2151 (1974). Once the reaction is complete, as measured by the consumption of the acyl azide, the compound of formula (ii) may be isolated by standard extractions and filtrations. If desired, the compound of formula (ii) may be further purified by chromatography or crystallization as appropriate.

(1R,3S)-3-(Carbonyl)cyclohexanecarboxylic acid, the compound of formula (i), is commercially available from Eastman Kodak or may be prepared as described in U.S. Pat. No. 6,028,213.

As the skilled artisan would appreciate, the compound of formula (i), dissolved in an appropriate solvent, is first treated with an appropriate azide and optionally a catalyst. Once the reaction is complete, as measured by the consumption of substrate, then an appropriate alcohol is added.

Appropriate solvents useful for the process of step (a) of the invention must be capable of dissolving a sufficient amount of the compound of formula (i) and then the corresponding azide for the reaction to proceed. Useful organic solvents include hexamethylphosphoramide, dimethylformamide, and toluene.

Suitable azides for this reaction include sodium azide, potassium azide, and the like. Preferably diphenylphosphoryl azide is the azide of choice. The skilled artisan would appreciate that diphenylphosphoryl azide will not require a catalyst. However, the use of sodium azide and potassium azide and the like may require a activating agent, such as ethyl chloroformaate or sulfuric acid. The substrate may need to be pretreated with the activating agent, such as the case with ethyl chloroformate, or may need to be added simultaneously. The skilled artisan would appreciate the potential for reaction at the ester site of the substrate, if the molecule is treated with the azide first as is the case in these circumstances.

Appropriate alcohols are lower alkyl alcohols such as methanol, ethanol, propanol, isopropanol, butanol, TMS-ethanol, benzyl, t-butanol, and the like. Methanol is the preferred alcohol.

The process of part (1) of step a) may be carried out over a large range of concentrations, from about 0.001 molar to about 2.0 molar of the azide, dependent upon the solubility of the particular azide in the chosen solvent. The reaction may also be performed on slurries of the azide so long as a sufficient amount of the azide is soluble in the solvent for the reaction to proceed. Preferably the process is performed at a concentration from about 0.1 molar to about 1.0 molar. A concentration of about 0.3 to about 0.4 molar is most preferred.

Additionally, from about 0.1 molar to about 3.0 molar of the alcohol can be used in part (2) of step (a). Preferably the process is performed at a concentration from about 1.5 molar to about 2.5 molar. A concentration of about 2.0 molar is most preferred.

Reactions of part (1) of step (a) may be performed between about 80° C. and about 130° C., preferably between about 100° C. and about 120° C. Most preferably the reactants are combined at temperature of about 20° C. to about 30° C., then heated to about 80° C. and about 120° C., the azide is added, and the reaction is stirred for about 0.5 to about 1.5 hours at reflux. For part (2) of step (a), the mixture from part (1) is allowed to cool to about 20° C. to about 100° C., preferably from about 70° C. to about 90° C. and then the appropriate alcohol is added. This reaction mixture is then warned to about 70° C. to about 90° C., if needed, for about 3 to about 24 hours, preferably about 75° C. to about 85° C. for about 8 to about 12 hours.

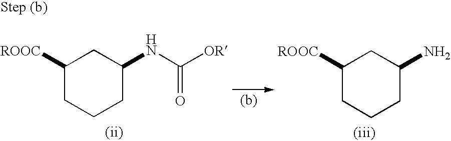

The deprotection process of step b) of the invention is performed by combining the compound of formula (ii) with an appropriate deprotecting agent in an appropriate reaction medium. Once the reaction is complete, as measured by consumption of the substrate, the resulting compound of formula (iii) is isolated by standard extractions and filtrations. If desired, the resulting compound of formula (iii) may be further purified by chromatography or crystallization as appropriate. Methods of removing an amine protecting group are well known in the art, for example, see T. W. Greene, “Protective Groups in Organic Synthesis”, John Wiley and Sons, New York, N.Y., 1991, Chapter 7 hereafter referred to as “Greene”.

Compounds useful as deprotecting agents, those compounds which cleave the protecting group from the compound of formula (ii), depends on the protecting group used, as the skilled artisan would appreciate. For example, a strong acid, such as trifluoroacetic acid or iodotrimethylsilane will remove a BOC protecting group.

Reaction media useful for the process of step (b) must be capable of dissolving a sufficient amount of the compound of formula (ii) for the reaction to proceed. Organic solvents useful as reaction media for the process of this invention depend upon the choice of deprotecting agent and may include tetrahydrofuran, acetonitrile or chlorocarbons.

Depending upon the choice of deprotecting agent, reactions of step (b) may be performed between about −30° and about 60° C. The skilled artisan will appreciate that the reaction rates will decrease as temperatures are lowered and increase as temperatures are elevated.

The process of step (b) may be carried out over a large range of concentrations, from about 0.001 molar to about 2.0 molar of the compound of formula (iii), dependent upon the solubility of the particular product in the chosen reaction medium. Preferably the process is performed at a concentration from about 0.01 molar to about 1.0 molar. A concentration of about 0.20 molar to about 0.50 molar is most preferred.

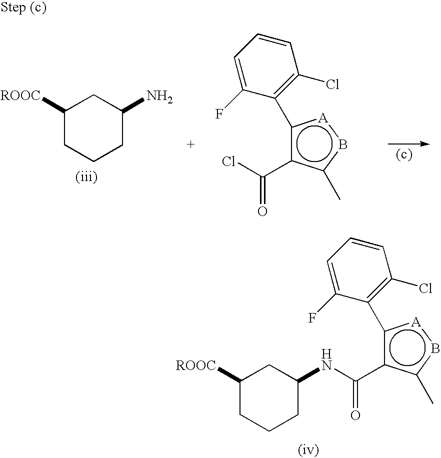

The process of step (c) may be performed by reacting the compound of formula (iii), in an appropriate medium, with (6-chloro-2-fluorophenyl)-methylisoxazole-4-carbonyl chloride to provide the compound of formula (iv). Once the reaction is complete, as measured by the consumption of the acid chloride, the resulting compound of formula (iv) may be isolated by standard extractions and filtrations. If desired, the compound of formula (iv) maybe further purified by chromatography or crystallization as appropriate.

3-(2-Chloro-6-fluorophenyl)-5-methylisoxazole-4-carbonyl chloride is commercially available. 5-(2-Chloro-6-fluorophenyl)-3-methylisoxazole-4-carbonyl chloride may be prepared as described in Scheme 4.

The order and manner of combining the reactants are not important and may be varied as a matter of convenience. The substrate and the acid chloride may first be combined and then the reaction medium added. Alternatively, the substrate may first be dissolved in an appropriate reaction medium and this solution added to a mixture of the acid chloride. Also, a solution of the substrate in an appropriate reaction medium may be added to a slurry of the acid chloride in the same reaction medium. Furthermore, a first slurry containing part of the reactants in an appropriate reaction medium may be added to a second slurry of the remaining reactants in an appropriate reaction medium as is desired or convenient. All of these methods are useful for the process of the present invention.

Reaction media useful for step (c) of the invention must be capable of dissolving a sufficient amount of the compound of formula (iii) products for the reaction to proceed. Organic solvents useful as reaction media for the process of this invention may include pyridine, triethylamine, 1:1 mixture of DMF and dichloromethane, mixture of tetrahydrofuran and water, dimethylformamide, and preferably tetrahydrofuran/water.

As the skilled artisan would appreciate, adding an aqueous base to the reaction mixture is preferable. Bases useful to the process of step (c) include 4-dimethylaminopyridine (DMAP) and preferably potassium carbonate.

The acid chloride is typically employed in an equimolar amount, relative to the amine, but a slight excess (about a 0.05 to about 0.15 molar excess) is acceptable.

Reactions of step (c) may be performed between about −30° and about 130° C. Preferably the reactants are combined between about 20° C. and about 30° C., then stirred for about 10 to about 14 hours. The skilled artisan will appreciate that the reaction rates will decrease as temperatures are lowered and increase as temperatures are elevated.

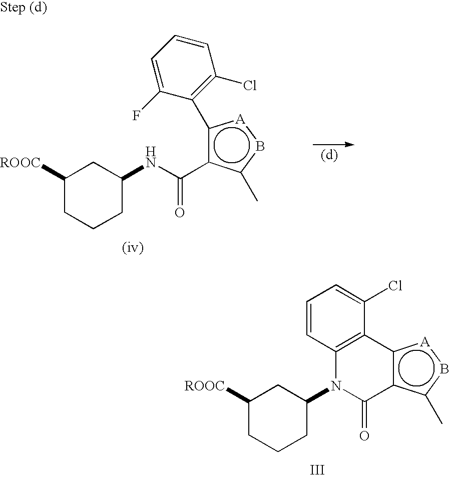

Step (d) of the reaction is performed by cyclizing the compound of formula (iv), in an appropriate solvent, in the presence of an appropriate catalyst, to form the compound of formula III. Once the reaction is complete, as measured by the consumption of the compound of formula (iv), the resulting compound of formula III may be isolated by standard extractions and filtrations. If desired, the compound of formula III may be further purified by chromatography or crystallization as appropriate.

The compound of formula III may be prepared by dissolving or suspending the compound of formula (iv) in a suitable solvent, preferably dimethylformamide, and adding a suitable base, including potassium methoxide, potassium tert-butoxide, potassium carbonate, sodium hexamethyldisilazane, and preferably potassium hexamethyldisilazane. The base is typically employed in a one to one ratio. However, as the skilled artisan would appreciate, a slight molar excess, usually in about a 1.1 to about a 3 fold molar excess relative to the compound of formula (iv), is acceptable.

The reactants are typically combined at a temperature from about 0° C. to about 100° C. The reactants are preferably combined at room temperature and the resulting solution is typically mixed for about 5 minutes to about 18 hours, preferably from about 15 minutes to about 3 hours.

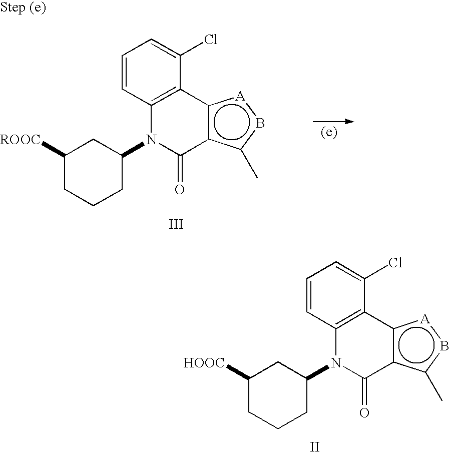

The process of step (e) of the reaction is performed by hydrolyzing the compound of formula III to form the compound of formula II. Once the reaction is complete, as measured by the consumption of the compound of formula m, the resulting compound of formula II may be isolated by standard extractions and filtrations. If desired, the compound of formula II may be further purified by chromatography or crystallization as appropriate.

The hydrolysis of the ester to the carboxylic acid is performed by standard techniques in the art, see for example Org Rxs 24 187 (1976) and Tetr 36 2409. Generally, the ester is treated with an appropriate aqueous base in an appropriate reaction medium.

Reaction media useful for the process of step (e) of the invention must be capable of dissolving a sufficient amount of the ester for the reaction to proceed. Organic solvents useful as reaction media for the process of this invention include dimethylformamide, diethyl ether, dimethoxyethane, and preferably tetrahydrofuran.

Suitable aqueous bases for this transformation include aqueous potassium hydroxide, lithium hydroxide, and preferably sodium hydroxide.

Reactions of step (e) may be performed between about −30° and about 100° C., preferably between about 50° C. and about 70° C. The skilled artisan will appreciate that the reaction rates will decrease as temperatures are lowered and increase as temperatures are elevated.

Compounds of formula xxx, where A is O and B is N, may be prepared by scheme

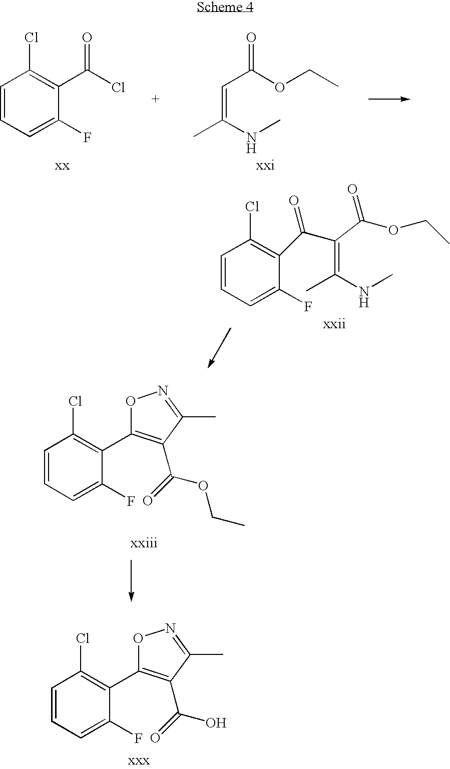

Compounds of formula xxii may be prepared by dissolving or suspending a compound of formula xxi and a suitable base in a suitable solvent and adding a compound of formula xx in a suitable solvent, dropwise. Toluene is a convenient solvent and is typically preferred. Triethylamine is the preferred base. The compound of formula xx is typically and preferably employed in an equimolar amount, relative to the compound of formula xxi, but a slight excess is acceptable.

The reactants are preferably combined at about 0° C. and the resulting solution is typically warmed to room temperature and mixed for from about 18 hours to about 24 hours.

The compound of formula xxii may then be converted to the compound of formula xxiii by dissolving or suspending a compound of formula xxii in a suitable acidic solvent and adding hydroxylamine hydrochloride. Glacial acetic acid is a convenient acidic solvent and is typically preferred. The ester group is then hydrolyzed to the corresponding carboxylic acid of formula xxx through standard procedures commonly employed in the art, see for example, Larock, pgs 981-985. The carboxylic acid of formula xxx may be converted to the corresponding acid chloride through standard procedures commonly employed in the art, see for example, Larock, pgs 963-964.

The reactants are preferably combined at about room temperature then heated to reflux for from about 30 minutes to about 60 minutes. Preferably the reaction is heated to reflux from about 40 to 45 minutes.

Compounds of formula xx and xxi are known in the art and, to the extent not commercially available, are readily synthesized by standard procedures commonly employed in the art.

The terms and abbreviations used herein have their normal meanings unless otherwise designated, for example, “°C.” refers to degrees Celsius; “N” refers to normal or normality; “mmol” refers to millimole or millimoles; “g” refers to gram or grams; “d” refers to density, “min.” refers to minutes, “mL” means milliliter or milliliters; “M” refers to molar or molarity; “HPLC” refers to high performance liquid chromatography; “mm” refers to millimeters; “cm” refers to centimeters; “nm” refers to nanometers; and “tᵣ” refers to retention time.

### PREPARATIONS AND EXAMPLES

### Preparation 1

### 1,3-cyclohexanedicarboxylic Acid

To a suspension of isophthalic acid (500 g, 3 mol) in methanol (2.8 l) was added 5% Rhodium-on-alumina catalyst (50 g) and acetic acid (150 ml). The reaction mixture was shaken under hydrogen (50 psi) at room temperature overnight. The mixture was filtered through celite. To this solution was added fresh 5% Rhodium-on-alumina catalyst (25 g), and the mixture was shaken under 50 psi of hydrogen for another 24 hours. The final reaction mixture was filtered through celite. The solution was concentrated under vacuum to give 493 g of the title compound as a white powder (96.3% yield). m.p. 163-165° C.

¹H NMR of 2: (300 MHz, CDCl₃) δ 9.00-10.00 (sb, 2H), 2.95 (m, 0.5H), 2.20-2.40 (m, 2.5H), 1.90-2.10 (m, 3H), 1.78 (m, 1H), 1.58 (t, 1H), 1.39 (d, 2H).

### Preparation 2

### 3-Oxabicyclo[3.3. 1]nonane-2,4-dione

A solution of dicyclohexylcarbodiimide (200 g, 1.16 mol) in CH₂Cl₂ (1000 ml) was added dropwise to a suspension of compound from preparation 1 (257 g, 1.25 mol) in CH₂Cl₂ (550 ml), and the mixture was stirred at room temperature for 4 hours. The precipitated dicyclohexylurea was filtered and washed several times with cold CH₂Cl₂ (200 ml×3). The combined organic layer was concentrated to give a white solid, which was suspended in MTBE (900 ml). This solid was collected by filtration, washed with MTBE (250 ml), and dried under house vacuum to give the title compound (137 g). The filtrate was concentrated to a residue, which was suspended in MTBE (250 ml) to give another 31 g of anhydride. The total yield was 168 g (94%). m.p. 138-140° C.

¹H NMR: (300 MHz, CDCl₃) δ 3.06 (m, 2H), 2.25 (d, 1H), 2.11 (m, 2H), 1.65-1.86 (m, 4H), 1.40-1.60 (m, 1H).

### Preparation 3

### cis-1,3-Cyclohexanedicarboxylic Acid Diethyl Ester

To a solution of compound from preparation 2 (31 g, 0.2 mol) in ethanol (anhydrous, 310 ml) was added p-toluenesulfonic acid monohydrate (1.9 g, 10 mmol, 0.05 equiv.) and triethyl orthoformate (50 ml, 0.3 mol). The reaction mixture was stirred at 60° C. overnight. The volatiles were stripped and the residue was diluted with ethyl acetate (250 ml), washed with water (120 ml) and brine (100 ml), and dried over MgSO₄. After filtration and evaporation, the residue was purified by chromatography. Eluting the column with 10% ethyl acetate in hexane afforded the title compound (40 g, 87.7% yield).

¹H NMR: (500 MHz, CDCl₃) δ 4.11 (q, J=7.0 Hz, 4H), 2.29 (dt, 2H), 2.11 (dd, 1H), 1.97 (m, 2H), 1.98 (m, 1H), 1.53 (q, J=12.5 Hz, 2H), 1.30-1.40 (m, 2H), 1.25 (t, J=7.0 Hz, 6H).

### Preparation 4

### 1,3-Cyclohexanedicarboxylic Acid, Monoethyl Ester (1R, 3S)

To a suspension of compound from preparation 3 (40 g, 17.5 mmol) in pH 7.2 phosphate buffer [0.2 M] (1.2 l) was added lipase AY30 (Amano, 16.7 g). The mixture was stirred vigorously at room temperature for 30 hours. The mixture was acidified with 10-15% HCl to pH<2, and extracted with ethyl acetate (500 ml×2). The combined organic solution was washed with aqueous 10% Na₂CO₃ (100 ml×2) and water (100 ml×2). The combined aqueous layers were washed again with ethyl acetate (150 ml) and then acidified with 10-15% HCl to pH<2. The acidified aqueous was then extracted with ethyl acetate (150 ml×3). The combined organic solution was dried over MgSO₄. After filtration and concentration the title compound (35.6 g, 100% yield) was obtained.

¹H NMR (500 MHz, CDCl₃) δ 4.12 (q, J=7.0 Hz, 2H), 2.20-2.40 (m, 3H), 1.85-2.05 (m, 3H), 1.5 (q, 2H), 1.35 (m, 2 H), 1.24 (t, J=7.0 Hz, 3H). [α]_{D} +3.2°, [α]₃₆₅ +10.4° (c, 0.434; CHCl₃). [α]_{D}+3.0°, [α]₃₆₅ +9.6° (c, 0.532; CH₃OH).

### Preparation 5

### Ethyl-[3-N-(methylcarbamate)-cyclohexyl]-carboxylate (1R, 3S)

A solution of a compound from preparation 4 (73 g, 365 mmol) in toluene (750 ml) was heated to reflux using a Dean-Stark trap to separate trace amounts of water. After collecting about 10 ml of water, the mixture was cooled down to about 40-50° C. To this mixture was added triethylamine (56 ml, 0.4 mol), and diphenylphosphoryl azide (86.5 ml, 0.4 mol). The reaction mixture was stirred at 110° C. for 60 min, cooled to 70° C., and methanol (64 g, 2 mol ) was added with stirring. After addition, the final reaction mixture was then heated to 85° C. overnight. After cooling to room temperature, the mixture was diluted with ethyl acetate (700 ml) and washed with water (500 ml). The aqueous layer was extracted with ethyl acetate (500 ml×2). The combined organic solution was washed again with water (500 ml) and brine (500 ml). After drying over MgSO₄ and concentration under reduced pressure, the title compound was obtained as a colorless oil (86 g, 100%).

¹H NMR: (300 MHz, CDCl₃) δ 4.60 (sb, 1H), 4.13 (q, 2H), 3.65 (s, 3H), 3.50 (sb, 1H), 2.38 (t, 1H), 2.23 (d, 1H), 2.00-1.80 (m, 3H), 1.24 (t, 3H), 1.12-0.95 (m, 1H ).

### Preparation 6

### Ethyl-((1R,3S)-3-{[3-(2-chloro-6-fluorophenyl)-5-methylisoxazol-4-yl]carbonyl-amino}cyclohexyl)-carboxylate

To a solution of compound from preparation 5 (86 g, 365 mmol) in CH₂Cl₂ (750 ml) was added iodotrimethylsilane (100 g, 500 mmol) in one portion, at room temperature. The reaction mixture was stirred for 2 hours at ambient temperature, cooled to 0-5° C., and methanol (50 ml) was added. After stirring 15 minutes, the solution was concentrated under reduced pressure. The residue was dissolved in THF (1 l). To this solution was added water (0.5 l), potassium carbonate (138 g, 1 mol), and a solution of 3-(6-fluoro-2-chlorophenyl)-5-methylisooxazole-4-carboxyryl chloride (110 g, 0.4 mol) in 250 ml THF, dropwise. After the addition, the reaction mixture was heated to room temperature and stirred for 12 hours. TBF was removed under house vacuum, water (250 ml) was added, and the mixture was extracted with ethyl acetate (500 ml×3). The combined organic solution was washed with saturated sodium thiosulfate (150 ml), water (500 ml), brine (500 ml) and then dried over MgSO₄. After filtration and evaporation under vacuum, the residue was purified by recrystallization from MME (250 ml). Repeating this recrystallization procedure three times provided the title compound (122.7 g, 82.5% yield) as a white powder.

[α]_{D} −13.30°, [α]₃₆₅ −51.90°(c, 0.54; CHCl₃).

IR: νₘₐₓ (film) 3429, 3011, 2940, 1725, 1662, 1187 cm¹.

¹H NMR: (300 MHz, CDCl₃) δ 7.51 (m, 1H), 7.39 (dd, 1H), 7.20 (dt, 1H), 5.15 (d, J=4.8 Hz, 1H), 4.08 (q, J=3.6 Hz, 2H), 3.80 (m,.5H), 2.77 (s, 3H), 2.36 (tt, 1H), 2.05 (m, J=4.8 Hz, 1H), 1.88 (d, J=7.5 Hz, 1H), 1.77 (m, 2H), 1.34 (q, J=7.8 Hz, 1H), 1.23 (t, J=3.6 Hz, 3H), 1.21 (m, 1H), 0.93 (q, J=7.2 Hz, 1H), 0.72 (dq, 1H).

¹³C NMR (300 MHz, CDCl₃) δ 174.8, 174.0, 162.4, 159.8, 152.8, 132.7, 132.5, 126.0, 125.8, 115.0, 114.9, 60.4, 47.3, 41.9, 34.6, 32.0, 28.0, 23.7, 14.1, 12.9. M.S. m/z 409(M⁺, 100%).

Anal. Calcd. for C₂₀H₂₂ClFN₂O₄: C, 58.7542; H, 5.4237; N, 6.8516. Found: C, 58.70; H, 5.26; N, 6.79.

### Preparation 7

### Ethyl [(1R,3S)-3-(9-chloro-3-methyl-4-oxo-5H-isoxazolo[4,3-c]quindin-5-yl)-cyclohexyl]-carboxylate

To a solution of compound from preparation 6 (78 g, 190 mmol) in DMF (750 ml) was added a solution of KHMDS ([0.5M], 400 ml, 200 mmol). The temperature was kept at 25° C. by using an ice-bath. After the addition was complete, the reaction mixture was analyzed by TLC (silica gel, 50% EtOAc in hexane) and found to be complete. Water (1 l) was added and the mixture was extracted with EtOAc (800 ml×3). The combined organic solution was washed with 1N HCl (250 ml), water (250 ml), brine (250 ml), dried over MgSO₄ and concentrated to give a residue which was purified by recrystallization from MTBE (500 ml) to afford 66 g of the title compound as a light yellow powder (89.0% yield).

[α]_{D}−14.1° (c,1.298; CHCl₃).

IR: νₘₐₓ (film) 3030, 1720, 1670, 1220 cm⁻¹.

¹H NMR: (300 MHz, CDCl₃) δ 7.48 (m, 2H), 7.35 (m, 1H), 4.12 (q, J=7.0 Hz, 2H), 2.90 (s, 3H), 2.80 (m, 1H), 2.52 (m, 2H), 2.03 (q, 4H), 1.81 (d, 1H), 1.52 (m, 2H), 1.23 (t, J=7.0 Hz, 3H).

¹³C NMR (300 MHz, CDCl₃) δ 174.3, 174.1, 159.1, 155.5, 133.8, 130.8, 125.2, 114.6, 112.8, 60.5, 43.7, 31.5, 28.3, 28.0, 25.3, 14.2, 12.8.

M.S. m/z 389 (M⁺+1, 100%).

### Preparation 8

### (1R,3S)3-(9-chloro-3-methyl-4-oxo-5H-isoxazolo[4,3-c]quinolin-5-yl)-cyclohexyl Carboxylic Acid

To a solution of compound from preparation 7 (62 g, 160 mmol) in THF (600 ml) was added 5N aqueous sodium hydroxide (120 ml) at room temperature. The reaction mixture was heated to 60° C. for 15 hours with stirring. After cooling to room temperature, water (750 ml) was added and the mixture was washed with ethyl acetate (500 ml). The aqueous phase was separated and acidified with 15% HCl to pH<2. The aqueous phase was then extracted with methylene chloride (1000 ml×3). The combined organic extracts were washed again with water (500 ml), brine (500 ml), and dried over MgSO₄. After filtration and evaporation under vacuum, the dark brown residue was suspended in MTBE (1000 ml), and refrigerated overnight. The mixture was filtered to afford 55.45 g (96.4%) of bright yellow product.

[α]_{D}−13.1°, [α]₄₃₆−23.3° (c, 0.58; CHCl₃).

IR: νₘₐₓ (Film) 3200, 2936, 1726, 1643, 1595, 1173 cm⁻¹.

¹H NMR: (300 MHz, CDCl₃) δ 7.45 (t, 1.5H), 7.34 (d, 1.5H), 4.4 (sb, 1H), 2.90 (s, 3H), 2.82 (sb, 1H), 2.58 (sb, 2H), 2.00-2.20 (m, 4H), 1.82 (m, 1H), 1.53 (db, 2H).

¹³C NMR (300 MHz, DMSO) δ 175.7, 173.8, 158.1, 154.8, 131.8, 124.7, 115.5, 111.4, 107.7, 47.5, 42.3, 33.3, 30.9, 27.9, 27.6, 25.3, 24.5, 24.4, 12.3.

M.S. m/e 361 (M⁺, 50%), 225 (100%).

Anal. Calcd. for C₁₈H₁₇ClN₂O₄: C, 59.9226; H, 4.7493; N, 7.7642. Found: C, 60.00; H, 5.01; N, 7.87.

### Preparation 9

### 2-methyl [(1R,3S)-3-(9-chloro-3-methyl4oxo-5H-isoxazolo[4,3-c]quinolin-5-yl)-cyclohexyl] Carbamate

To a suspension of compound from preparation 8 (55.4 g, 154 mmol) in toluene (1 l) was added triethylamine (23.7 ml, 170 mmol), and diphenylphosphoryl azide (36.5 ml, 170 mmol). The reaction mixture was stirred at 110° C. for 2 hours during which time a solution formed. The solution was cooled to 80° C. and methanol (25 g, 0.77 mol) was added with stirring. The solution was warmed to 85° C. for 22 hours. After cooling to room temperature, the toluene was removed under reduced pressure and the residue was dissolved in dichloromethane (3 l) and washed with water (1 l). The aqueous phase was extracted with dichloromethane (1 l×2) and the combined organic solution was washed again with water (500 ml) and brine (500 ml). After drying over MgSO₄, the solution was concentrated under vacuum. The crude product was purified by crystallization (CH₂Cl₂/MTBE, 0.5 ½ l ) to afford the title compound (46.6 g, 78.2%). [α]_{D}+49.2°, [α]₃₆₅ +263.3° (c, 0.56; CHCl₃). The filtrate was concentrated to a residue, which was purified by chromatography to obtain a second crop of product (5.1 g). The total yield was 86.8%.

IR νₘₐₓ (Film) 3410, 3020, 2950, 1710, 1670, 1220 cm⁻¹.

¹H NMR: (300 MHz, CDCl₃) δ 7.45 (m, 1.5H), 7.33 (m, 1.5H), 4. 63 (sd, J=4.5 Hz, 1H), 3.62 (sb, 1H), 3.61 (s, 3H), 2.87 (s, 3H), 2.59 (sb, 2H), 2.15 (d, J=6.3 Hz, 1H), 2.03 (d, J=6.9 Hz, 1H), 1.95 (d, J=8.1 Hz, 1H), 1.79 (sb, 2H), 1.45 (d, 1H), 1.24 (m, J=6.3 Hz, 1H).

¹³C NMR (300 MHz, CDCl₃) δ 173.9, 159.2, 155.9, 155.5, 133.8, 131.0, 125.3, 114.1, 112.7, 62.9, 57.7, 50.0, 36.0, 32.5, 27.9, 23.8, 17.7, 12.7-1.5.

M.S. m/z 476 (M⁺, 25%).

Anal. Calcd. for C₂₃H₃₀ClN₃O₄Si: C, 58.0307; H, 6.3521; N, 8.8268. Found: C, 57.95; H, 6.76; N, 8.44.}

### Preparation 10

### (1R,3S)3-(9-Chloro-3-methyl-4-oxo-5H-isoxazolo[4,3-c]quinolin-5-yl)-cyclohexyl Methyl Alcohol

To a solution of compound from preparation 8 (4.4 g, 12.2 mmol) in THF (45 ml) was added borane-methyl sulfide complex (2.0 M solution in THF, 12.5 ml, 25 mmol) dropwise at 0° C. When the addition was complete, the reaction mixture was allowed to warm to room temperature and was stirred for one hour. Methanol (10 ml) was added slowly (gas generated) with stirring. The reaction mixture was then poured into ice-water (60 ml) and extracted with ethyl acetate (100 ml×3). The combined organic solution was washed with 1N HCl (50 ml), brine (50 ml), and dried over MgSO₄. After filtration and evaporation under vacuum, the yellow title compound (4.34 g, 100%) was obtained.

[α]_{D} +5.1°, [α]₃₆₅ +39.6° (c, 1.05; CHCl₃).

¹H NMR: (300 MHz, CDCl₃) δ 7.45 (t, 2H), 7.34 (d, 1H), 3.58 (m, 2H), 2.90 (s, 3H), 2.59 (m, 1H), 2.40 (m, 1H), 2.00 (m, 1H), 1.92 (m, 1H), 1.84 (m, 2H), 1.75 (m, 2H), 1.50 (m, 1H), 1.12 (m, 1H).

¹³C NMR: (250 MHz, CDCl₃) δ 174.0, 159.2, 155.5, 133.7, 130.8, 125.1, 114.6, 112.7, 67.8, 59.2, 41.1, 32.3, 29.0, 28.4,25.5, 12.8.

M.S.: m/z 347 (M⁺+1).

HRMS calcd. for C₁₈H₂₀ClN₂O₃: 347.1162. Found: 347.1177.

### Preparation 11

### (1R,3S)3-(9-chloro-3-methyl4-oxo-5H-isoxazolo[4,3-c]quinolin-5-yl)-clohexyl Methyl Alcohol Mesylate

To a stirred solution of compound from preparation 10 (4.32 g. 12.5 mol) in pyridine (25 ml) was added DMAP (10 mg) and methanesulfonyl chloride (1.16 ml, 15 mmol, 1.2 equiv) and the resulting mixture was stirred at room temperature for 1.5 h. Water (100 ml) was added, and the mixture was extracted with ethyl acetate (150 ml×2). The combined organic extracts were washed again with brine (100 ml), dried (MgSO₄) and concentrated. The residue was suspended in MTBE (25 ml) and filtered to obtain the title compound as a solid (4.65 g, 87.8%).

[α]_{D} +8.71°, [α]₃₆₅ +58.7° (c, 0.358; CHCl₃).

¹H NMR: (300 MHz, CDCl₃) δ 7.45 (t, 1.5H), 7.34 (d, 1.5H), 4.00-4.20 (m, 2H), 3.00 (s, 3H), 2.90 (s, 3H), 2.59 (br s, 1H), 2.42 (br s, 1H), 2.00 (m, 4H), 1.84 (m, 21H), 1.50 (m, 1H), 1.12 (m, 1H).

### Preparation 12

### (1R,3S)3-(9-chloro-3-methyl-4-oxo-5H-isoxazolo[4,3-c]quinolin-5-yl)-cyclohexyl Methyl Azide

A mixture of a compound from preparation 1 l (4.6 g, 10.8 mmol), sodium azide (2.15 g, 33 mmol) and DMF (45 ml) was heated to 60° C. and stirred for 24 h. After cooling to room temperature, the mixture was poured into 150 ml of ice-water and extracted with MTBE (200 ml×2). The combined organic extracts were washed with brine (150 ml) and dried over MgSO₄. After filtration and concentration, the residue was chromatographed on silica gel using 30% ethyl acetate/hexane to give the title compound as a white powder (3.82 g, 95%).

[α]_{D} +17.2°, [α]₃₆₅ +105.7° (c, 0.864; CHCl₃).

¹H NMR: (300 MHz, CDCl₃) δ 7.45 (t, 1.5H), 7.34 (d, 1.5H) 3.24 (m, 2H), 2.90 (s, 3H), 2.59 (br s, 1H), 2.42 (br s, 1H), 2.00 (m, 1H), 1.84 (m, 5H), 1.50 (m, 1H), 1.12 (m, 1H).

### Preparation 13

### (1R,3S)3-(9-chloro-3-methyl-4-oxo-5H-isoxazolo[4,3-c]quinolin-5-yl)-cyclohexyl Methyl Amine

To a solution of a compound from preparation 12 (2.0 g, 5.4 mmol) in ethyl acetate (25 ml) was added 5% Pd/C catalyst (200 mg). The reaction mixture was shaken under hydrogen (50 psi) at room temperature for 2 days. The mixture was filtered through celite and the filtrate was concentrated under vacuum to give the title compound as a solid (1.85 g, 99.2%).

¹H NMR: (300 MHz, CDCl₃) δ 7.45 (t, 1.5H), 7.34 (d, 1.5H), 2.90 (s, 3H), 2.62 (m, 2H), 2.59 (br s, 1H), 2.42 (br s, 1H), 2.00 (m, 1H), 1.84 (m, 4H), 1.50 (m, 2H), 1.12 (m, 1H).

### Preparation 14

### Ethyl-((1R,3S)-3-{[5-(2-chloro-6-fluorophenyl)-3-methylisoxazol4yl]carbonyl-amino}cyclohexyl)-carboxylate

To a solution of a compound from preparation 4 (42 g, 182 mmol) in CH₂Cl₂ (400 ml) was added iodotrimethylsilane (36.5 ml, 255 mmol) in one portion at room temperature. The reaction mixture was stirred for 2 hours at ambient temperature and then cooled down to 0-5° C. To this mixture was added methanol (50 ml) and the mixture was stirred 15 minutes and concentrated under reduced pressure. The residue was dissolved in THF (300 ml), and water (150 ml) and potassium carbonate (62 g, 0.45 mol) were added. To the resulting mixture was slowly added a solution of 5-(2-chloro-6-fluorophenyl)-3-methyl-isooxazole-4-carboxyl chloride (50 g, 182 mmol) in 50 ml of THF. After the addition, the reaction mixture was allowed to warm to room temperature and was stirred for 12 hours. THF was removed under house vacuum, water (150 ml) was added and the mixture was extracted with ethyl acetate (500 ml×2). The combined organic solution was washed with water (250 ml), brine (250 ml) and dried over MgSO₄. After filtration and evaporation under vacuum, the residue was purified by silica gel chromatography (30% ethyl acetate in hexane) to obtain the title compound (62.57 g, 84.3% yield) as a white powder.

[α]_{D}−1.8°, [α]₃₆₅ +8.0° (c, 0.54; CHCl₃).

IR: νₘₐₓ (film) 3428, 3016, 2938, 2861, 1722, 1665, 1521, 1452, 1195 cm⁻¹.

¹H NMR: (300 MHz, CDCl₃) δ 7.55 (m, 1H), 7.39 (dd, 1H), 7.20 (dt, 1H), 5.19 (d, J=4.8 Hz, 1H), 4.08 (q, J=3.6 Hz, 2H), 3.85 (m, 1H), 2.55 (s, 3H), 2.38 (m, 1H), 2.10 (m, J=4.8 Hz, 1H), 1.88 (d, J=7.5 Hz, 1H), 1.77 (m, 2H), 1.34 (q, J=7.8 Hz, 1H), 1.22 (t, J=3.6 Hz, 3H), 1.05 (m, 1H), 0.85 (dq, 1H).

M.S.: m/z 409 (M⁺, 100%).

HRMS calcd. for C₂₀H₂₃ClFN₂O₄: 409.1330. Found: 409.1328

Anal. Calcd. for C₂₀H₂₂ClFN₂O₄: C, 58.7542; H, 5.4237; N, 6.8516; Cl, 8.6712. Found: C, 58.66; H, 5.43; N, 6.77; Cl, 8.77.

### Preparation 15

### Ethyl[(1R,3S)-3-(9-chloro-3-methyl4-oxo-5H-isoxazolo[4,5-c]quindin-5-yl)-cyclohexyl]-carboxylate

To a 0-5° C. solution of a compound from preparation 14 (61.5 g, 0.15 mol) in DMF (500 ml) was added a solution of KHMDS (340 ml, [0.5M], 0.17 mol) in toluene. After the addition was complete, the mixture was stirred at ambient temperature for 15 minutes and analyzed by TLC (silica gel, 50% EtOAc in hexane), which indicated completion of the reaction (TLC showed a minor by-product spot along with the major product spot). Water (1 l) was added and the mixture was extracted with EtOAc (800 ml×3). The combined organic extracts were washed with water (250 ml), brine (250 ml), dried over MgSO₄ and concentrated to a residue. The residue was purified by recrystallization from MTBE (200 ml) to obtain 28.12 g of the title compound. The filtrate was concentrated and purified by silica gel chromatography to obtain an additional 12.4 g of the title compound (40.52 g total, 69.3% yield).

[α]_{D}−20.1°, [α]₃₆₅−72.5° (c, 0.68; CHCl₃).

¹H NMR: (300 M CDCl₃) δ 7.50 (m, 1.5H), 7.35 (m, 1.5H), 5.80 (sb, 0.5H), 4.30 (sb, 0.5H), 4.12 (q, J=7.0 Hz, 2H), 2.80-3.00 (m, 1H), 2.66 (s, 3H), 2.52 (m, 2H), 2.03 (q, 3H), 1.81 (d, 1H), 1.52 (m, 2H), 1.20 (t, J=7.0 Hz, 3H).

¹³C NMR: (300 MHz, CDCl₃) δ 174.3, 174.1, 166.0, 159.0, 158.2, 131.9, 130.8, 124.8, 114.6, 111.3, 60.8, 44.0, 31.6, 28.3, 28.0, 25.6, 14.5, 11.2.

M.S.: m/z 389 (M⁺+1, 100%).

HRMS calcd. for C₂₀H₂₁ClN₂O₄: 389.1268. Found: 389.1271.

Anal. Calcd. for C₂₀H₂₁ClN₂O₄: C, 61.7771; H, 5.4435; N, 7.2041; Cl, 9.1173. Found: C, 61.65; H, 5.37; N, 7.13; Cl, 9.10.

### Preparation 16

### (1R,3S)3-(9-chloro-3-methyl4oxo-5H-isoxazolo[4,5-c]quinolin-5-yl)-cyclohexyl Carboxylic Acid

To a solution of a compound from preparation 15 (40 g, 103 mmol) in THF (350 ml) was added 5N aqueous sodium hydroxide (88 ml) at room temperature. The reaction mixture was warmed to 60° C. and stirred for 15 hours. After cooling to room temperature, water (500 ml) was added and the mixture was washed with ethyl acetate (500 ml). The aqueous phase was separated and acidified with 15% HCl to pH<2. The precipitate was collected by filtration and washed with ethyl acetate (250 ml). The filtrate was extracted with ethyl acetate (500 ml) and the combined organic solution was washed again with water (200 ml), brine (200 ml), and dried over MgSO₄. After filtration and evaporation under vacuum, the residue was combined with the precipitate obtained from acidification of the reaction mixture and suspended in MTBE (500 ml). The suspension was filtered to afford the title compound as a bright yellow product (36.0 g, 100%).

[α]_{D}−10.9°, [α]₃₆₅−44.4° (c, 0.358; 1N NaOH aqueous).

¹H NMR: (300 MHz, DMSO−d₆) δ 7.95 (br s, 1H), 7.65 (t, 1H), 7.45 (t, 1H), 5.60 (br s, 0.3H), 4.60 (br s, 0.7H), 2.82 (br s, 1H), 2.60 (s, 3H), 2.58 (br s, 2H), 2.00-2.20 (m, 4H), 1.82 (m, 1H), 1.53 (db, 2H).

¹³C NMR: (300 MHz, DMSO) δ 175.8, 165.0, 157.9, 157.5, 132.2, 129.6, 124.3, 115.0, 109.7, 57.7, 42.2, 30.7, 27.9, 27.5, 24.4, 22.8, 10.3.

M.S.: m/e 361 (M⁺+1, 100%);

HRMS calcd. for C₁₈H₁₈ClN₂O₄: 361.0955. Found: 361.0937.

### Preparation 17

### (1R,3S)3-(9-chloro-3-methyl-4-oxo-5H -isoxazolo[4,5-c]quinolin-5-yl)-cyclohexyl Methyl Alcohol

To a suspension of a compound from preparation 16 (37.52 g, 0.104 mmol) in THF (350 ml) was added borane-methyl sulfide complex in 140 ml of TBF (26 ml, 0.27 mol) dropwise at 0° C. After the addition, the reaction mixture was stirred at 0-5° C. for one hour. TLC (3:1 EtOAc/hexane) indicated the completion of the reaction. Methanol (50 ml) was added slowly (gas generated) with stirring, followed by aqueous 10% HCl (50 ml). After stirring for 15 minutes, the reaction mixture was poured into ice water (250 ml) and extracted with ethyl acetate (350 ml×2). The combined organic solution was washed with brine (300 ml), dried over.MgSO₄ and concentrated under vacuum. The crude product was purified by silica gel chromatography (EtOAc/hexane, 1:1) to give the title compound (32.5 g, 90%).

[α]_{D}+2.5°, [α]₃₆₅+72° (c, 0.436; CHCl₃).

¹H NMR: (300 MHz, CDCl₃) δ 7.55 (t, 1.5H), 7.34 (d, 1.51H), 5.80 (br s, 0.3H), 4.39 (br s, 0.7H), 3.58 (m, 2H), 2.67 (s, 3H), 2.59 (br s, 1H), 1.60-2.00 (m, 7H), 1.50 (m, 1H), 1.12 (m, 1H).

¹³C NMR: (300 MHz, CDCl₃) δ 165.9, 159.0, 158.0, 131.7, 131.2, 124.5, 113.7, 111.1, 67.7, 41.1, 32.1, 28.8, 25.5, 10.7.

M.S.: m/e 347 (M⁺+1).

HRMS calcd. for C₁₈H₁₉ClN₂O₃: 347.1137. Found: 347.1162.

Anal. Calcd. for C₁₈H₂₀ClN₂O₃: C, 62.3386; H. 5.5221; N, 8.0773; Cl, 10.2224. Found: C, 62.07; H, 5.38; N, 7.92; Cl, 10.15.

### Preparation 18

### (1R,3S)3-(9-chloro-3-methyl4-oxo-5H-isoxazolo[4,5-c]quinolin-5-yl)-cyclohexyl Methyl Alcohol Mesylate

To a stirred solution of a compound from preparation 17 (32.25 g. 93.2 mmol) in pyridine (270 ml) was added DMAP (20 mg) and methanesulfonyl chloride (7.9 ml, 102 mmol, 1.1 equiv.). The mixture was stirred at room temperature for 1.5 hours. Water (500 ml) and EtOAc/MTBE (500 ml, 1:1) were added causing the product to precipitate. The solid was collected by filtration, washed with MTBE (150 ml) and dried under vacuum to provide 29.35 g as a white powder. The filtrate was washed again with brine (300 ml), dried (MgSO₄), and concentrated. The residue was suspended in MTBE (50 ml) and filtered to give a second crop of the title compound (9.5 g, total yield: 38.8 g, 99.3%).

[α]_{D} +2.1°, [α]₃₆₅ +90.1° (c, 0.564; CHCl₃).

IR: ₘₐₓ (Film) cm^{31 1} 3018, 2935, 2855, 1675, 1360, 1175, 958.

¹H NMR: (300 MHz, CDCl₃) δ 7.52 (t, 2H), 7.39 (d, 1.5H), 4.05-4.20 (m, 2H), 3.00 (s, 3H), 2.70 (br s, 1H), 2.69 (s, 3H), 2.05 (m, 2H), 2.00-1.80 (m, 4H), 1.50 (m, 1H), 1.12 (m, 1H).

¹³C NMR: (300 MHz, CDCl₃) δ 175.7, 158.9, 158.0, 131.8, 131.4, 113.6, 111.1, 73.6, 38.2, 37.3, 31.6, 28.4, 27.9, 25.1, 10.7.

M.S.: m/e425 (M⁺+1).

HRMS calcd. for C₁₉H₂₁ClN₄O₅S: 425.0938. Found: 425.0936.

Anal. Calcd. for C₁₉H₂₁ClN₂O₅S: C, 53.7087; H. 4.9817; N, 6.5928; Cl, 8.3437. Found: C, 54.08; H, 5.04; N, 6.70; Cl, 8.31.

### Preparation 19

### (1R,3S)3-(9-chloro-3-methyl4oxo-5H-isoxazolo[4,5-c]quinolin-5-yl)-cyclohexyl Methyl Azide

A mixture of a compound from preparation 18 (38.2 g, 90 mmol), sodium azide (21 g, 0.32 mol), and DMF (350 ml) was heated to 60° C. with stirring for 20 hours. After cooling to room temperature, the mixture was poured into ice-water (500 ml) and extracted with EtOAc (500 ml×2). The combined organic phases were washed with brine (250 ml) and dried over MgSO₄. After filtration and evaporation, the residue was chromatographed on silica gel using 30% ethyl acetate/hexane to give the title compound as a white powder (28.2 g, 84.4%).

[α]_{D}+12.1°, [α]₃₆₅ +167.4° (c, 0.614; CHCl₃).

IR: νₘₐₓ (Film) cm⁻¹ 3019, 2101, 1675, 1562, 1438.

¹H NMR: (300 MHz, CDCl₃) δ 7.51 (t, 2H), 7.36 (d, 1H), 4.30 (br s, 1H), 3.26 (m, 2H), 2.70 (s, 3H), 2.65 (br s, 1H), 2.02 (m, 1H), 2.00 (m, 1H), 1.85 (m, 5H), 1.50 (m, 1H), 1.16 (m, 1H).

¹³C NMR: (300 MHz, CDCl₃) δ 159.0, 158.0, 131.8, 131.3, 124.6, 113.7, 111.1, 59.6, 57.2, 38.7, 32.9, 29.4, 28.5, 25.4, 10.7.

M.S.: m/e 372 (M⁺+1).

Anal. Calcd. for C₁₈H₁₈ClN₅O₂: C, 58.1451; H, 4.8795; N, 18.8348; Cl, 9.5347. Found: C, 58.44; H, 4.95; N, 18.75; Cl, 9.65.

### Preparation 20

### 1,3-Cyclohexanedicarboxylic Acid

To a suspension of isophthalic acid (5.0 g, 30.1 mmol) in 45 ml of acetic acid was added a slurry of 0.1 g of platinum oxide in 5 ml of acetic acid. The resulting mixture was stirred under 50 psi of hydrogen at 25° C. for 16 hours. NMR analysis (DMSO-d₆) at this time showed complete reduction of starting material. The reaction mixture was filtered through Celite and the filter cake was rinsed with methanol. The combined filtrate and washes were concentrated under reduced pressure, using heptane to azeotropically remove residual acetic acid. Trituration of the resultant semi-solid with heptane and filtration of the precipitate provided 4.92 g (95%) of the title compound as a white powder.

mp: 163-165° C.

¹H NMR (300 MHz, CDCl₃) δ 9.00-10.00 (br s, 2H), 2.95 (m, 0.5H), 2.20-2.40 (m, 2.5H), 1.90-2.10 (m, 3H), 1.78 (m, 1H), 1.58 (t, 1H), 1.39 (d, 2H).

### Preparation 21

### 3-Oxabicyclo[3.3.1]nonane-2,4-dione

A suspension of 1,3-cyclohexanedicarboxylic acid (490 g, 2.88 mol) in acetic anhydride (1500 ml) was heated to 140° C., refluxing for 2 hours. Acetic anhydride was then removed with distillation (oil bath 180° C.). To the residue was added acetic anhydride (1000 ml) and refluxed at 140° C. for 1 hour. The acetic anhydride was removed again with distillation (under house vacuum, about 50° C.). After crystals appeared, the mixture was cooled to room temperature and MTBE (400 ml) was added. The mixture was then cooled to 0-5° C. The crystals were filtered, washed with MTBE (250 ml), and dried under house vacuum to give the title compound (382 g). The filtrate was concentrated to a residue and suspended in MTBE (100 ml) to give the second crop of the title compound (14.0 g). The total yield was 396 g (90.5%).

Mp 138-140° C.

¹H NMR: (300 MHz , CDCl₃) δ 3.06 (m, 2H), 2.25 (d, 1H), 2.11 (m, 2H), 1.65-1.86 (m, 4H), 1.40-1.60 (m, 1H).

### Example 1

### Benzoyl [(1R,3S)-3-(9-chloro-3-methyl-4-oxo5H-isoxazolo[4,3c]quinolin-5-yl)-cyclohexyl] Amide

To a solution of the compound from preparation 9 (160 mg, 0.41 mmol) in CH₂Cl₂ (3 ml) was added iodotrimethylsilane (124 mg, 0.62 mmol) in one portion, at room temperature. The reaction mixture was stirred for 2 hours at ambient temperature and cooled to 0-5° C. Methanol (1 ml) was added and the mixture was stirred for 15 minutes and concentrated under reduced pressure. The residue was dissolved in TBF (2 ml). To this solution was added water (1 ml), potassium carbonate (210 mg, 1.5 mmol), and benzoyl chloride (60 mml, 0.5 mmol). The resultant mixture was stirred at room temperature for 2 hours. TBF was removed under house vacuum, water (15 ml) was added, and the mixture was extracted with ethyl acetate (15 ml×2). The combined organic extracts were washed with saturated sodium thiosulfate (10 ml), brine (10 ml) and dried over MgSO₄. After filtration and evaporation under vacuum, the residue was purified by silica gel chromatography (35% EtOAc/hexane) to obtain the title compound (145 mg, 81.0% yield) as an off-white powder.

¹H NMR: (300 MHz, DMSO-d₆) δ 8.40 (d, 1H), 7.82 (m, 4H), 7.62 (t, 1H), 7.43 (m, 4H), 4.5 (br s, 1H), 4.05 (m, 1H), 2.82 (s, 3H), 2.78 (m, 1H), 1.88 (m, 4H), 1.71 (m, 2H), 1.60 (m, 1H), 1.41 (m, 1H).

### Example 2

### 4-Fluoro-3-pyridyl-carboxyl-(1R,3S)3-(9-chloro-3-methyl-4-oxo-5H-isoxazolo[4,3-c]quinolin-5-yl)-cyclohexyl Methyl Amide

To a solution of a compound from preparation 13 (466 mg, 1.35 mmol) in THF/H₂O (5 ml/2.5 ml) was added potassium carbonate (690 mg, 5 mmol) and 2-fluoro-pyridine-4-carboxyl chloride (240 mg, 1.5 mmol) at room temperature. After the addition, the reaction mixture was stirred for 2 hours at ambient temperature. Water (50 ml) was added and the mixture was extracted with dichloromethane (50 ml×2). The combined organic extracts were washed with water (35 ml), brine (35 ml) and dried over MgSO₄. After filtration, the filtrate was concentrated to a residue (720 mg) which was purified by silica gel chromatography (ethyl acetate/dichloromethane, 1:2) to give the title compound (450 mg, 71.0%) as a white powder.

[α]_{D} +37°.[α]₄₃₆ +87.0° (c, 0.70; CHCl₃).

IR: νₘₐₓ (Film) cm⁻¹3456, 3000, 2935, 2861, 1670, 1631, 1596, 1479, 1266.

¹H NMR: (500 MHz, CDCl₃) δ 8.58 (d, J=2.0 Hz, 1 H), 8.22 (dt, J₁=2.5 Hz, J₂=8.0 Hz, 1H), 7.45 (t, 1.5H), 7.34 (d, 1.5H), 6.98 (bb, J₁=2.5 Hz, J₂=8.5 Hz, 1H), 6.33 (br s, 1H), 3.40 (m, 2H), 2.89 (s, 31H), 2.58 (br s, 2H), 2.10 (m, 2H), 1.83-1.95 (m, 4H), 1.50 (qb, 1H), 1.17 (dq, 1H).

³C NMR: (300 MHz, CDCl₃) δ 174.0, 166.5, 164.6, 163.3, 159.1, 146.7, 140.9, 140.8, 133.7, 130.9, 128.6, 125.2, 114.5, 112.6, 109.9, 109.4, 59.2, 45.9, 38.5, 33.4, 29.7, 28.7, 25.4,12.8.

M.S.: m/z 469 (M⁺+100%).

Anal. Calcd. for C₂₄H₂₂ClFN₄O₃: C, 61.4749; H, 4.7290; N, 11.9481; Cl, 7.5606; F, 4.0515. Found: C, 61.24; H, 4.70; N, 11.55; Cl, 7.50; F, 4.46.

### Example 3

### 4-fluoro-3-pyridyl-carboxyl-(1R, 3S) 3-(9-chloro-3-methyl-4-oxo-5H-isoxazolo[4,5-c]quinolin-5-yl)-cyclohexyl Methyl Amide

To a solution of the compound from preparation 19 (2.0 g, 5.4 mmol) in THF (120 ml) was added triphenylphosphine (10.5 g, 40 mmol) and water (25 ml). The mixture was stirred under nitrogen at room temperature overnight. To the reaction mixture was added 15% aqueous HCl (15 ml) and the mixture was stirred for 30 minutes. The mixture was then poured into water (200 ml) and washed with ethyl acetate/MTBE (150 ml/50 ml). Some product precipitated and was suspended in the aqueous layer. The organic layer was separated and washed with water (70 ml). The combined aqueous phases (about 270 ml) were washed again with ethyl acetate/NMTE (100 ml/50 ml). To the aqueous suspension was added THF (270 ml), K₂CO₃ (41 g, 0.3 mol) and 2-fluoropyridine4-carboxyl chloride (6.4 g, 40 mmol) at room temperature. After the addition, the reaction mixture was stirred for 20 hours. The mixture was extracted with ethyl acetate (250 ml×2) and the combined organic extracts were washed with brine (200 ml), and dried over MgSO₄. After filtration, the filtrate was concentrated to give (16.5 g, 99.7%) of a white powder. The product was further purified by recrystallization from methanol to afford the title compound (14.0 g, 85%).

[α]_{D} +36.6°, [α]₄₃₆ +281° (c, 0.40; CHCl₃).

IR: νₘₐₓ (Film) cm⁻¹ 3446, 3017, 2929, 1674, 1596, 1479, 1437, 1267.

¹H NMR: (500 MHz, CDCl₃) δ 8.59 (d, J=2.0 Hz, 1H), 8.25 (dt, J₁=2.5 Hz, J₂=8.0 Hz, 1H), 7.45 (t, 1.5H), 7.34 (d, 1.5H), 6.99 (bb, J=2.5 Hz, J=8.5 Hz, 1H), 6.40 (sb, 1H), 5.79 (sb, 0.3H), 4.35 (sb, 0.7H), 3.40 (m, 2H), 2.70 (sb, 2H), 2.67 (s,3H), 2.02 (m, 1H), 1.85-1.95 (m, 3 H), 1.82 (m, 1H), 1.50 (m, 1H), 1.17 (dq, 1H).

¹³C NMR: (300 MHz, DMSO-d₆) δ 165.6, 165.0, 163.6, 162.1, 157.9, 157.5, 147.3, 147.1, 141.4, 141.2, 132.2, 129.6, 128.8, 128.7, 124.3, 109.7, 109.5, 108.9, 45.1, 32.7, 5 29.5,28.0,24.6, 10.3.

M.S.: m/e 469 (M⁺+1).

HRMS calcd. for C₂₄H₂₃ClFN₄O₃: 469.1442. Found: 469.1456.

### Example 4

### N-[(1R,3S)-3-(9-chloro-3-methyl-4-oxo-5H-isoxazolo[4,3-c]quinolin-5-yl)-cyclohexylmethyl]-nicotinamide

To a solution of (1R,3S)3-(9-chloro-3-methyl-4-oxo-5H-isoxazolo[4,3-c]quinolin-5-yl)-cyclohexyl methyl amide (700 mg, 2.0 mmol) in 35 mL of dichloromethane was added 440 mg (2.4 mmol) of nicotinoyl chloride hydrochloride, 0.85 mL (6.0 mmol) of triethylamine and 5 mg of 4-dimethylaminopyridine. The reaction mixture was stirred overnight at ambient temperature, then washed with 1 N hydrochloric acid. The aqueous layer was extracted with 20% isopropanol/chloroform. The combined organics were washed with saturated sodium carbonate, brine, dried over sodium sulfate, filtered and concentrated to dryness. The residue was chromatographed on silica gel using methanol/chloroform as eluent to yield 740 mg (82%) of the desired isomer as a white solid.

¹H-NMR is consistent with structure.

MS (ion spray) 451.1 (M+1).

Anal. Calc'd for C₂₄H₂₃ClN₄O₃0.1CHCl₃.

Theoretical: C, 62.54; H, 5.03; N, 12.10%.

Found: C, 62.71; H, 5.01; N, 12.04%.

### Example 5

### 6-Chloro-N-[(1R,3S)-3-(9-chloro-3-methyl4oxo-5H-isoxazolo[4,3-c]quinolin-5-yl)-cyclohexylmethyl]-nicotinamide

To a solution of 5-[3-(aminomethyl)cyclohexyl]-9-chloro-3-methyl-5-hydroisoxazolo[4,3-c]quinolin-4-one-HCl (86 mg, 0.22 mmol) in 20 mL of NN-dimethylformamide was added 62 μL (0.45 mmol) of triethylamine, 43 mg (0.27 mmol) of 6-chloronicotinic acid, 36 mg (0.27 mmol) of 1-hydroxy-7-azabenzo-triazole, 51 mg (0.27 mmol) of 1-(3-dimethylamino-propyl)-3-ethyl-carbodiimide hydrochloride and 5 mg of 4-dimethylaminopyridine. The reaction mixture was stirred overnight at ambient temperature and concentrated to dryness. The residue was partitioned between chloroform and saturated sodium bicarbonate. The mixture was washed with saturated sodium bicarbonate, water, brine, dried over sodium sulfate, filtered and concentrated to dryness. The residue was chromatographed on silica gel using methanol/chloroform as eluent and concentrated to dryness. The residue was slurried in ether/hexanes and concentrated to dryness to yield 77 mg (71%) of the desired isomer as a white foam.

¹H-NMR is consistent with structure.

MS (ion spray) 485.1 (M+).

Anal. Calc'd for C₂₄H₂₂Cl2N₄O₃0.1CHCl₃.

Theoretical: C, 58.21; H, 4.48; N, 11.27%.

Found: C, 58.15; H, 4.12; N, 10.95%.

The compounds of formula I are inhibitors of MRP1. Thus, the compounds of the invention may be used to inhibit any neoplasm having intrinsic and/or acquired resistance, conferred in part or in total by MRP1, to an oncolytic or oncolytics. In other words, treatment of such a neoplasm with an effective amount of a compound of this invention will cause the neoplasm to be more sensitive to chemotherapy that was rendered less efficacious by MRP1.

Vincristine, epirubicin, daunorubicin, doxorubicin, and etoposide are oncolytics that are substrates of MRP1. See Cole, et. al., “Pharmacological Characterization of Multidrug Resistant MRP-transfected Human Tumor Cells”, Cancer Research, 54:5902-5910, 1994. Since MRP1 is ubiquitous in mammals, particularly humans, Nooter, K, et. al., “Expression of the Multidrug Resistance-Associated Protein (NP) Gene in Human Cancers”, Clin. Can. Res., 1:1301-1310, (1995), chemotherapy whose goal is to inhibit a neoplasm employing any of those agents has the potential to be rendered less efficacious by MRP1. Thus, neoplasms of the bladder, bone, breast, lung(small-cell), testis, and thyroid and more specific types of cancer such as acute lymphoblastic and myeloblastic leukemia, Wilm's tumor, neuroblastoma, soft tissue sarcoma, Hodgkin's and non-Hodgkin's lymphomas, and bronchogenic carcinoma may be inhibited with a combination of one or more of the above oncolytics and a compound of this invention.

The biological activity of the compounds of the present invention was evaluated employing an initial screening assay, which rapidly and accurately measured the activity of the tested compound in inhibiting MRP1 or MDR1. Assays useful for evaluating this reversing capability are well known in the art. See, e.g., T. McGrath, et al., Biochemical Pharmacology, 38:3611, 1989; D. Marquardt and M.S. Center, Cancer Research, 52:3157, 1992; D. Marquardt, et al., Cancer Research, 50:1426, 1990; and Cole, et. al., Cancer Research, 54: 5902-5910, 1994.

Assay for Reversal of MRP1 -Mediated Doxorubicin Resistance and MDR1-Mediated Vincristine Resistance:

HL60/Adr and HL60/Vinc are continuous cell lines, which were selected for doxorubicin and vincristine resistance respectively by culturing HL60, a human acute myeloblastic leukemia cell line, in increasing concentrations of doxorubicin or vincristine until a highly resistant variant was attained.

HL60/Adr and HL60/Vinc cells were grown in RPMI 1640 (Gibco) containing 10% fetal bovine serum (FBS) and 50 μg/fl GENTAMICIN™ (Sigma). Cells were harvested; washed twice with assay medium (same as culture media); counted; and diluted to 1×10⁵ cells/ml in assay medium. One hundred microliters of cells were aliquoted into wells of a 96 well tissue culture plate. Two columns of each 96 well plate served as a negative control and received assay medium containing no cells.

Test compounds and reference compounds were dissolved in dimethyl sulfoxide (DMSO) at a concentration of 5 mM. Samples were diluted in assay medium and 25 μl of each test compound was added to 8 wells. Assay standards were run in quadruplicate. Assay media was added to half of the wells and doxorubicin to the other half of the wells to achieve a final volume of 150 μl per well.

The plates were incubated at 37° C. for 72 hours in a humidified incubator with a 5% carbon dioxide atmosphere. Cell viability and vitality was measured by oxidation of a alamarBlue™ fluorescent dye using standard conditions. The plates were incubated for 3 hours at 37° C. Fluorescence was determined using 550 nm excitation and 590 nm emission using a microtitre plate reader.

The ability of a test compound to reverse the resistance of HL60/Adr and HL60/Vinc cells to doxorubicin was determined by comparison of the absorbance of the wells containing a test compound in addition to the oncolytic (doxorubicin) with the absorbance of wells containing the oncolytic without a test compound. Controls were used to eliminate background and to ensure the results were not artifactual. The results of the assay are expressed as percent inhibition of cell growth. The oncolytic alone at the tested concentration minimally inhibits the growth of HL60/Adr or HL60/Vinc cells.

Representative compounds of formula I demonstrated a significant effect in reversing the MRP1 multiple drug resistance. Many of the compounds showed very significant enhancement of activity in combination with the oncolytic agent as opposed to the oncolytic agent alone. In addition, a large majority of the compounds tested displayed a significant degree of selective inhibition of the HL60/Adr cell line over the HL60/Vinc>cell line.

When administering an oncolytic in practicing the methods of this invention, the amount of oncolytic employed will be variable. It should be understood that the amount of the oncolytic actually administered will be determined by a physician, in the light of the relevant circumstances, including the condition to be treated, the chosen route of administration, the actual oncolytic administered, the age, weight, and response of the individual patient (mammal), and the severity of the patient's symptoms. Of course, the amount of oncolytic administered should be decided and closely monitored by that patient's physician. After deciding on the oncolytic or oncolytics to employ, “The Physician's Desk Reference®”, published by Medical Economics Company at Montvale, N.J. 07645-1742, is a helpful resource to the physician in deciding on amounts of the oncolytic to administer and is updated annually.

Preferred formulations, and the methods of this invention employing those formulations, are those which do not contain an oncolytic. Thus, it is preferred to administer the compounds of this invention separately from the oncolytic. The oncolytics mentioned in this specification are commercially available and may be purchased in pre-formulated forms suitable for the methods of this invention.

The compounds of formula I alone, or optionally in combination with an oncolytic, are usually administered in the form of pharmaceutical formulations. These formulations can be administered by a variety of routes including oral, rectal, transdermal, subcutaneous, intravenous, intramuscular, and intranasal. Such formulations are prepared in a manner well known in the pharmaceutical art and comprise at least one active compound of formula I.

The present invention also includes methods employing pharmaceutical formulations, which contain, as the active ingredient, the compounds of formula I, and optionally an oncolytic, associated with pharmaceutical carriers. In making the formulations of the present invention the active ingredient(s) is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders.

In preparing a formulation, it may be necessary to mill the active compound(s) to provide the appropriate particle size prior to combining with the other ingredients. If the active compound(s) is substantially insoluble, it ordinarily is milled to a particle size of less than 200 mesh. If the active compound(s) is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The formulations of the invention can be formulated so as to provide quick, sustained or delayed release of the active ingredient after administration to the patient by employing procedures known in the art.

The formulations are preferably formulated in a unit dosage form, each dosage containing from about 5 to about 100 mg, more usually about 10 to about 30 mg, of each active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The compounds of formula I are effective over a wide dosage range. For example, dosages per day normally fall within the range of about 0.5 to about 30 mg/kg of body weight. In the treatment of adult humans, the range of about 1 to about 15 mg/kg/day, in single or divided dose, is especially preferred. However, it will be understood that the amount of the compound actually administered will be determined by a physician, in the light of the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

For preparing solid formulations such as tablets the principal active ingredient(s) is mixed with a pharmaceutical excipient to form a solid preformulation composition containing a homogeneous mixture of a compound of the present invention. When referring to these preformulation compositions as homogeneous, it is meant that the active ingredient(s) is dispersed evenly throughout the formulation so that the formulation may be readily subdivided into equally effective unit dosage forms such as tablets, pills and capsules. This solid preformulation is then subdivided into unit dosage forms of the type described above containing from 0.1 to about 500 mg of the active ingredient of the .present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The novel formulations which are liquid forms may be incorporated for administration orally or by injection and include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Formulations for inhalation or insufflation include solutions and suspensions in pharmaceutical, aqueous or organic solvents, or mixtures thereof, and powders. The liquid or solid formulations may contain suitable pharmaceutical excipients as described supra. Preferably the formulations are administered by the oral or nasal respiratory route for local or systemic effect. Compositions in preferably pharmaceutical solvents may be nebulized by use of inert gases. Nebulized solutions may be breathed directly from the nebulizing device or the nebulizing device may be attached to a face mask, tent, or intermittent positive pressure breathing machine. Solution, suspension, or powder formulations may be administered, preferably orally or nasally, from devices that deliver the formulation in an appropriate manner.

The following formulation examples are illustrative only and are not intended to limit the scope of the invention in any way. “Active ingredient(s)” means a compound according to formula I or a pharmaceutical salt or solvate thereof optionally with one or more oncolytics.

### Formulation Example 1

Hard gelatin capsules containing the following ingredients are prepared:

| | Quantity |
|---|---|
| Ingredient | (mg/capsule) |
| Active ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| | |
|---|---|
| Ingredient | Weight % |

| | |
|---|---|
| Active ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity | |
|---|---|---|
| Ingredient | (mg/tablet) | |
| Active ingredient | 30.0 | mg |
| Starch | 45.0 | mg |
| Microcrystalline cellulose | 35.0 | mg |
| Polyvinylpyrrolidone (as 10% solution in water) | 4.0 | mg |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | |
|---|---|
| | Quantity |
| Ingredient | (mg/capsule) |

| | | |
|---|---|---|
| Active ingredient | 40.0 | mg |
| Starch | 109.0 | mg |
| Magnesium stearate | 1.0 | mg |
| Total | 150.0 | mg |

The active ingredient, cellulose, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 ml dose are made as follows:

| Ingredient | Amount | |
|---|---|---|
| Active ingredient | 50.0 | mg |
| Xanthan gum | 4.0 | mg |
| Sodium carboxymethyl cellulose (11%)/Microcrystalline | 50.0 | mg |
| cellulose (89%) | | |
| Sucrose | 1.75 | g |
| Sodium benzoate | 10.0 | mg |
| Flavor and Color | q.v. | |
| Purified water to | 5.0 | ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

Capsules, each containing 15 mg of medicament, are made as follows:

| | |
|---|---|
| | Quantity |
| Ingredient | (mg/capsule) |

| | | |
|---|---|---|
| Active ingredient | 15.0 | mg |
| Starch | 407.0 | mg |
| Magnesium stearate | 3.0 | mg |
| Total | 425.0 | mg |

The active ingredient, cellulose, starch, and magnesium stearate are blended, passed through a No. 20 mesh U.S. sieve, and filled into hard gelatin capsules in 425.0 mg quantities.

### Formulation Example 9

An intravenous formulation may be prepared as follows:

| Ingredient | Quantity | |
|---|---|---|
| Active ingredient | 250.0 | mg |
| Isotonic saline | 1000 | ml |

### Formulation Example 10

A topical formulation may be prepared as follows:

| | |
|---|---|
| Ingredient | Quantity |

| | | |
|---|---|---|
| Active ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### Formulation Example 11

Sublingual or buccal tablets, each containing 10 mg of active ingredient, may be prepared as follows:

| | Quantity |
|---|---|
| Ingredient | Per Tablet |
| Active ingredient | 10.0 mg |
| Glycerol | 210.5 mg |
| Water | 143.0 mg |
| Sodium Citrate | 4.5 mg |
| Polyvinyl Alcohol | 26.5 mg |
| Polyvinylpyrrolidone | 15.5 mg |
| Total | 410.0 mg |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the active ingredient is slowly admixed. The homogenous mixture is poured into forms made of an inert material to produce a drug-containing diffusion matrix having a thickness of about 2-4 mm. This diffusion matrix is then cut to form individual tablets having the appropriate size.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical formulation to the brain, either directly or indirectly. Direct techniques usually involve placement of a drug delivery catheter into the host's ventricular system to bypass the blood-brain barrier. One such implantable delivery system, used for the transport of biological factors to specific anatomical regions of the body, is described in U.S. Pat. No. 5,011,472, issued Apr. 30, 1991, which is herein incorporated by reference.

Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions, which can transiently open the blood-brain barrier.

We claim:

1. A process for preparing (1R,3S)-3-(9-chloro-3-methyl-4-oxo-5H-(isoxazoloquinolin-5-yl))cyclohexanecarboxylic acid and esters thereof, as represented by formulas II and III, wherein R is a lower alkyl group, benzyl, aryl, or heterocycle, and A and B are N or O, provided that when A is N, B is O, or when A is O, B is N: 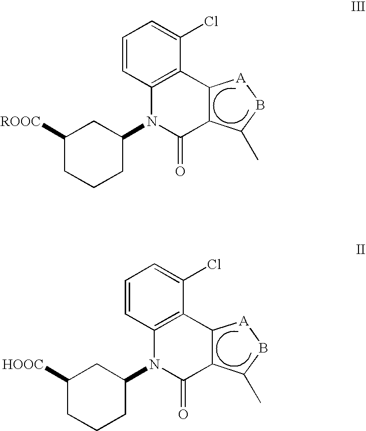
comprising the steps of:
(a) treating, in an appropriate solvent, a compound of formula (i), wherein R is as defined above: 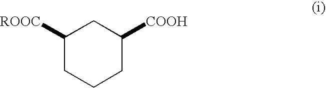
with an appropriate azide and subsequently an appropriate alcohol to form a compound of formula (ii), wherein R is defined above and R' is a lower alkyl group, benzyl, aryl, or heterocycle: 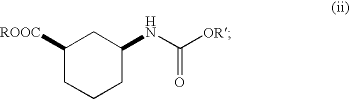
(b) deprotecting the compound of formula (ii), in an appropriate reaction medium, with an appropriate deprotecting agent to provide a compound of formula (iii), wherein R is as defined above: 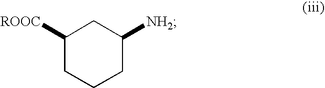
(c) reacting the compound of formula (iii), in an appropriate solvent, with (6-chloro-2-fluorophenyl)-methylisoxazole4-carbonyl chloride to provide the compound of formula (iv), wherein R, A, and B are as defined above: 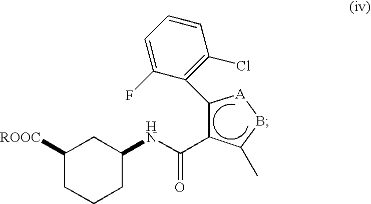
(d) cyclizing the compound of formula (iv), in an appropriate solvent, in the presence of an appropriate catalyst, to form a compound of formula III, wherein R, A, and B are as defined above: 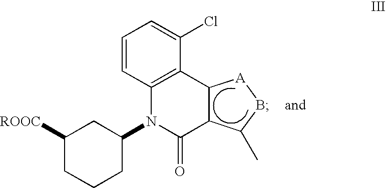
(e) optionally, hydrolyzing the compound of formula III to form a compound of formula II, wherein R, A, and B are as defined above:

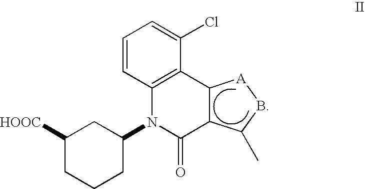

2. A process for converting the compounds of formulas It or im to form a compound of formula I(a): 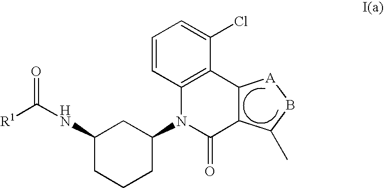
wherein:
the stoichiometry is substituted 1R,3S)-3-(9-chloro-3-methyl-4-oxo-5H-(isoxazoloquinolin-5-yl) cyclohexane;
a is 0, 1, 2, 3, or 4;
b is 0, 1, or 2;
u is 0, 1, 2, 3, or 4;
A is N or O; when A is N, B is O, or when A is O, B is N;
R¹ is independently at each occurrence C₁-C₆ alkyl, C₁-C₆ alkoxy, (C₁-C₄ alkoxy)-aryl, (C₁-C₄ alkoxy)-heterocycle, (C₁-C₄ alkoxy)-SiCH₃, optionally substituted (C₁-C₄ alkyl)-(C₃-C₈ cycloalkyl), optionally substituted (C₁-C₄ alkyl)-aryl, optionally substituted aryl, diphenylmethyl, optionally substituted (C₁-C₄ alkyl)-CO-aryl, optionally substituted (C₁-C₄ alkyl)-heterocycle, optionally substituted heterocycle, optionally substituted (C₁-C₄ alkyl)-phenoxy, (CH₂)ₐS(O)_{b}R², (CH₂)ₐC(R³)(R⁴)N(R⁵)(R⁶), (CH₂)ₐC(R³)(R⁴)O(R⁷), (CH₂)ₐC(R³)(R⁴)S(R⁷), or NR⁸R⁹;
R² is independently at each occurrence hydrogen or C₁-C₆ allyl;
R³ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, or optionally substituted heterocycle;
R⁴ is independently at each occurrence hydrogen, C₁-C₆ alkyl, optionally substituted C₃-C₈ cycloalkyl, optionally substituted (C₁-C₄ alkyl)-aryl, optionally substituted aryl, optionally substituted heterocycle, (CH₂)ᵤ-(C₁-C₆ alkoxy), optionally substituted (CH₂)ᵤ-O-(C₃-C₈ cycloalkyl), optionally substituted (CH₂)ᵤ-(C₁-C₄ alkoxy)-aryl, optionally substituted (CH₂)ᵤ-O-aryl, optionally substituted (CH₂)ᵤ-O-heterocycle, (C₁-C₄ alkyl)-CO₂-(C₁-C₆ alkyl), optionally substituted (C₁-C₄ alkyl)-CO₂-(C₃-C₈ cycloalkyl), optionally substituted (C₁-C₄ alkyl)-CO₂-(C₁-C₄ alkyl)-aryl, optionally substituted (C₁-C₄ alkyl)-CO₂-aryl, optionally substituted (C₁-C₄ alkyl)-CO₂-heterocycle, or R9 and R12 can combine to form a C₃-C₈ cycloalkyl;
R⁵ is independently at each occurrence hydrogen, optionally substituted C₁-C₆ alkyl, optionally substituted aryl, optionally substituted heterocycle, or —COR¹¹; or R⁵ and R⁶, together with the nitrogen to which they are attached, combine to form an optionally substituted N-heterocycle;
R⁶ is independently at each occurrence hydrogen, C₁-C₆ alkyl, optionally substituted C₃-C₈ cycloalkyl, optionally substituted C₆-C₁₀ bicycloalkyl, optionally substituted (C₁-C₄ alkyl)-aryl, optionally substituted aryl, optionally substituted (C₁-C₄ alkyl)-heterocycle, optionally substituted heterocycle, C(O)OR¹⁰, SO₂R¹¹, C(O)R¹², or a moiety of the formula 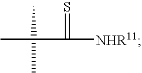
R⁷ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, optionally substituted heterocycle, optionally substituted (C₁-C₄ allyl)-heterocycle, optionally substituted C₁-C₆ alkoxy, optionally substituted (C₁-C₄ alkoxy)-aryl, optionally substituted (C₁-C₄ alkoxy)-heterocycle, (C₁-C₄ alkyl)-N(R²)(R²), or an amino acid ester;
R⁸ is independently at each occurrence hydrogen, C₁-C₆ alkyl, optionally substituted (C₁-C₆ alkyl)-aryl, optionally substituted aryl, or R⁸ and R⁹ combine to form ═CR²R¹³;
R⁹ is independently at each occurrence hydrogen, C₁-C₆ alkyl, C₁-C₄ alkoxy, optionally substituted C₃-C₈ cycloalkyl, optionally substituted C₆-C₁₀ bicycloalkyl, optionally substituted (C₁-C₄ alkyl)-aryl, optionally substituted aryl, optionally substituted (C₁-C₄ alkyl)-heterocycle, optionally substituted heterocycle, (C₁-C₄ alkyl)-OR¹⁰:
wherein the (C1-C4 alkyl) of the (C1-C4 alkyl)-OR10 is optionally substituted from 1 to 2 times with C1-C4 alkyl, optionally substituted aryl, optionally substituted heterocycle;
or R⁸ and R⁹, together with the nitrogen to which they are attached, combine to form an optionally substituted N-heterocycle;
R¹⁰ is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, or optionally substituted heterocycle;
R¹¹ is independently at each occurrence optionally substituted C₁-C₆ alkyl, optionally substituted aryl, optionally substituted (C₁-C₄ alkyl)-aryl, optionally substituted (C₁-C₄ alkyl)-heterocycle, or optionally substituted heterocycle;
R¹² is independently at each occurrence hydrogen, optionally substituted C₁-C₆ alkyl, optionally substituted C₃-C₈ cycloalkyl, optionally substituted (C₁-C₄ alkyl)-aryl, optionally substituted aryl, optionally substituted heterocycle, or optionally substituted (C₁-C₄ alkyl)-heterocycle; or a pharmaceutical salt thereof.
R¹³ is independently at each occurrence C₁-C₆ alkyl or optionally substituted (C₁-C₄ alkyl)-aryl;
or a pharmaceutically acceptable salt thereof;
comprising the following steps:
(a) reacting a compound of formula II, wherein A and B are as defined in claim 1: 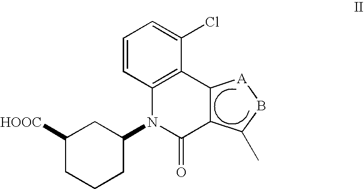
in an appropriate solvent, with an appropriate azide, then an appropriate alcohol, to provide a compound of formula (vi), wherein A and B are as defined in claim 1: 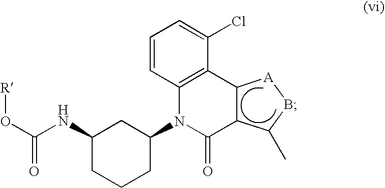
(b) deprotecting the compound of formula (vi), in an appropriate reaction medium, with an appropriate deprotecting agent to provide a compound of formula (vii), wherein A and B are as defined in claim 1: 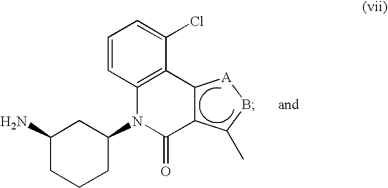
(c) acylating the compound of formula (vii), in a suitable solvent, with an appropriate acylating agent to provide a compound of formula I(a).

3. A process for converting the compounds of formulas II or III to form a compound of formula I(b), wherein R¹, A, and B are as defined in claim 2: 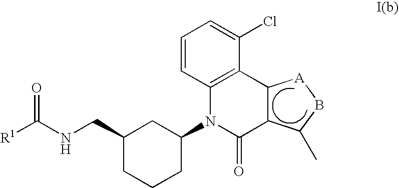
or a pharmaceutically acceptable salt thereof; comprising the following steps:
(a) reducing a compound of formula IL wherein A and B are as defined above: 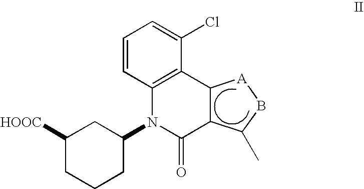
in an appropriate solvent, with an appropriate reducing agent to provide a compound of formula (x), wherein A and B are as defined above: 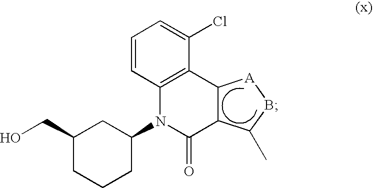
(b) reacting the compound of formula (x), in an appropriate solvent, with an appropriate nucleophile source to provide a compound of formula (xi), wherein A and B are as defined above and LG is a leaving group: 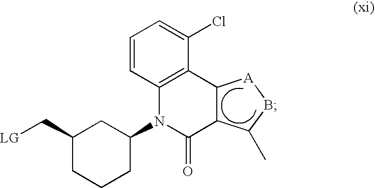
(c) reacting a compound of formula (xi), in an appropriate solvent, with an appropriate azide salt to provide a compound of formula (xii), wherein A and B are as defined above: 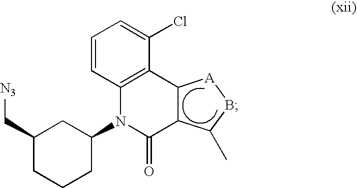
(d) reducing the compound of formula (xii), in an appropriate solvent, with an appropriate reducing agent to provide a compound of formula (xiv), wherein A and B are as defined above: 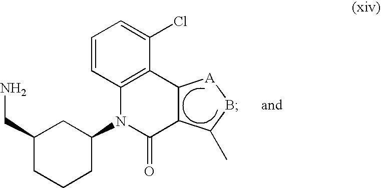
(e) acylating the compound of formula (xiv), in a suitable solvent, with an appropriate acylating agent to provide a compound of formula I(b).

4. A process for converting the compounds of formulas (i) to a compound of formula I(a), wherein R¹, A, and B are as defined in claim 2: 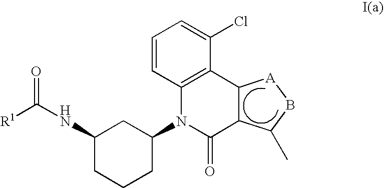
comprising the steps of:
(a) treating, in an appropriate solvent, a compound of formula (i), wherein R is as defined above: 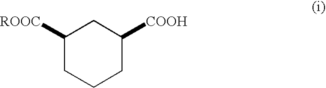
with an appropriate azide and subsequently an appropriate alcohol to form a compound of formula (ii), wherein R is defined above and R′ is a lower alkyl group, benzyl, aryl, or heterocycle: 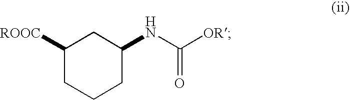
(b) deprotecting the compound of formula (ii), in an appropriate reaction medium, with an appropriate deprotecting agent to provide a compound of formula (iii), wherein R is as defined above: 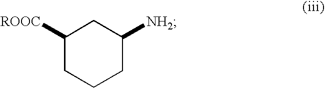
(c) reacting the compound of formula (iii), in an appropriate solvent, with (6-chloro-2-fluorophenyl)-methylisoxazole-4-carbonyl chloride to provide the compound of formula (iv), wherein R, A, and B are as defined in claim 2: 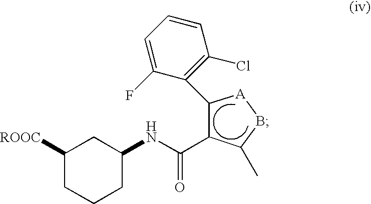
(d) cyclizing the compound of formula (iv), in an appropriate solvent, in the presence of an appropriate catalyst, to form a compound of formula III, wherein R, A, and B are as defined above: 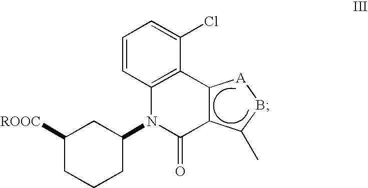
(e) hydrolyzing the compound of formula m to form a compound of 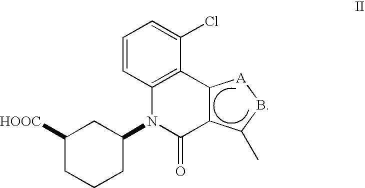
(f) reacting a compound of formula It in an appropriate solvent, with an appropriate azide, then an appropriate alcohol, to provide a compound of formula (vi), wherein A and B are as defined above: 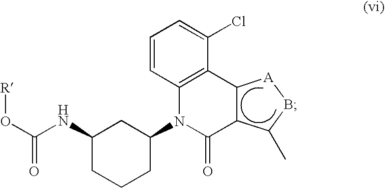
(g) deprotecting the compound of formula (vi), in an appropriate reaction medium, with an appropriate deprotecting agent to provide a compound of formula (vii), wherein R, A and B are as defined above: 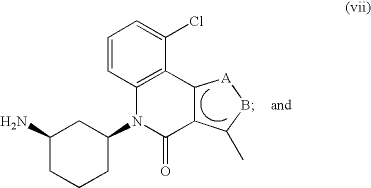
(h) acylating the compound of formula (vii), in a suitable solvent, with an appropriate acylating agent to provide a compound of formula I(a).

5. A process for converting the compounds of formulas (i) to a compound of formula I(b), wherein R¹, A, and B are as defined in claim 2: 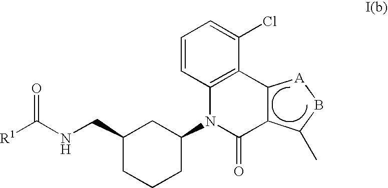
or a pharmaceutically acceptable salt thereof;
comprising the steps of:
(a) treating, in an appropriate solvent, a compound of formula (i), wherein R is as defined above: 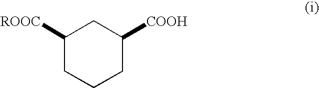
with an appropriate azide and subsequently an appropriate alcohol to form a compound of formula (ii), wherein R is defined above and R′ is a lower alkyl group, benzyl, aryl, or heterocycle: 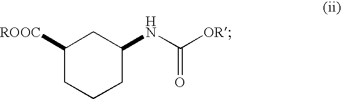
(b) deprotecting the compound of formula (ii), in an appropriate reaction medium, with an appropriate deprotecting agent to provide a compound of formula (iii), wherein R is as defined above: 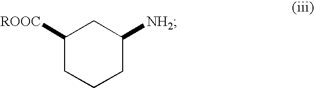
(c) reacting the compound of formula (iii), in an appropriate solvent, with (6-chloro-2-fluorophenyl)-methylisoxazole-4-carbonyl chloride to provide the compound of formula (iv), wherein R, A, and B are as defined above: 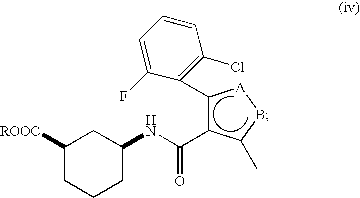
(d) cyclizing the compound of formula (iv), in an appropriate solvent, in the presence of an appropriate catalyst, to form a compound of formula III, wherein R, A, and B are as defined above: 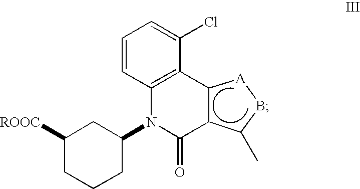
(e) hydrolyzing the compound of formula III to form a compound of formula II, wherein R, A, and B are as defined above: 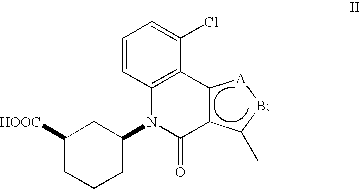
(f) hydrolyzing a compound of formula II in an appropriate solvent, with an appropriate reducing agent to provide a compound of formula (x), wherein A and B are as defined above: 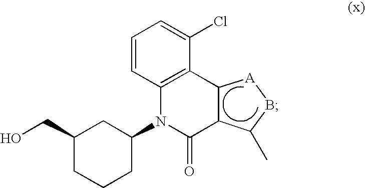
(g) reacting the compound of formula (x), in an appropriate solvent, with an appropriate nucleophile source to provide a compound of formula (xi), wherein A and B are as defined above and LG is leaving group: 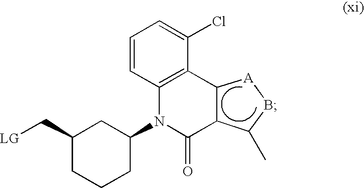
(h) reacting a compound of formula (xi), in an appropriate solvent, with an appropriate azide salt to provide a compound of formula (xii), wherein A and B are as defined above: 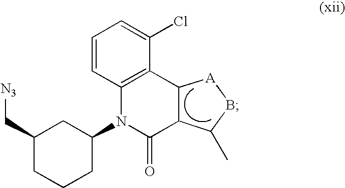
(i) reducing the compound of formula (xii), in an appropriate solvent, with an appropriate reducing agent to provide a compound of formula (xiv), wherein R, A and B are as defined above: 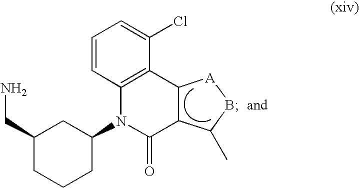
(j) acylating the compound of formula (xiv), in a suitable solvent, with an appropriate acylating agent to provide a compound of formula I(b).
